(19) United States
(12) Patent Application Publication
(10) Pub. No.: US 2024/0408100 A1
(43) Pub. Date: Dec. 12, 2024

(54) TREATMENT METHODS FOR SUBJECTS HAVING CANCER WITH A DYSREGULATED MAPK AND/OR PI3K PATHWAY

(71) Applicant: TAIHO PHARMACEUTICAL CO., LTD., Tokyo (JP)

(72) Inventors: Koji ICHIKAWA, Ibaraki (JP); Kimihiro ITO, Ibaraki (JP); Karim BENHADJI, Princeton, NJ (US)

(73) Assignee: TAIHO PHARMACEUTICAL CO., LTD., Tokyo (JP)

(21) Appl. No.: 18/699,669

(22) PCT Filed: Oct. 13, 2022
(86) PCT No.: PCT/JP2022/039011

§ 371 Date: Apr. 9, 2024

Related U.S. Application Data
(60) Provisional application No. 63/255,577, filed on Oct. 14, 2021.

(30) Foreign Application Priority Data
Oct. 13, 2021 (JP) 2021-168315

(51) Int. Cl. A61K 31/519; A61P 35/00

(52) U.S. Cl. CPC A61K 31/519; A61P 35/00

(57) ABSTRACT

A method of treating a subject with cancer having a dysregulated MAPK and/or PI3K pathway, whereby the subject is administered an effective amount of 4-(4-(3-((2-(tert-butylamino)ethyl)amino)-6-(5-(trifluoromethyl)-1,3,4-oxadiazol-2-yl)pyridin-2-yl)piperidin-1-yl)-5,5-dimethyl-5H-pyrolo[2,3-d]pyrimidin-6(7H)-one or a pharmaceutically acceptable salt thereof.

### BACKGROUND OF THE INVENTION

### Field of the Invention

The present invention relates to methods of treating cancers with a dysregulated MAPK and/or PI3K pathway.

### Description of the Related Art

The rat sarcoma (RAS) protein is a central regulator of cell proliferation and survival in normal cells and cancer cells. RAS activation activates effector pathways, most notably the mitogen-activated protein kinase (MAPK) pathway and the PI3 kinase (PI3K) pathway. See Pratilas CA et al., Targeting the mitogen-activated protein kinase pathway: physiological feedback and drug response. Clin Cancer Res. 2010;16(13):3329-3334. The signaling cascade of the MAPK pathway includes Ras/Raf/MEK/ERK/RSK, while the signaling cascade of the PI3K pathway includes Ras/PI3K/PTEN/AKT/mTOR/S6K. See Steelman LS et al., Roles of the Raf/MEK/ERK and PI3K/PTEN/Akt/mTOR pathways in controlling growth and sensitivity to therapy-implications for cancer and aging. Aging (Albany NY). 2011;3(3):192-222. The activities of these pathways are regulated through feedback loops, demonstrating signal transduction as nonlinear and highly interactive. See McCubrey JA et al., Mutations and deregulation of Ras/Raf/MEK/ERK and PI3K/PTEN/Akt/mTOR cascades which alter therapy response. Oncotarget. 2012;3(9):954-987.

Therapeutic single-agent pathway inhibition can be hindered by feedback loops on the reciprocal pathway. Additionally, the dysregulation of components of these cascades through activating/inactivating aberrations has been implicated in chemotherapeutic drug resistance and resistance to other pathway inhibitors, resulting in insufficient clinical response.

Neurofibromin 1 (NF1) is a gene that codes for neurofibromin, a GTPase-activating protein that is a negative regulator of the RAS pathways and, when aberrant, can be a significant driver in cancer. Mutations and deletions in NF1 are common in sporadic cancers and are associated with increased cancer risk and drug resistance. Additionally, NF1 gene germline mutations cause Neurofibromatosis type 1, and afflicted patients are at increased risk for several different types of adult cancers, including breast cancer. NF1 genetic variants are seen in 2-4% of breast cancers and NF1 shallow deletions have been observed in 25% of sporadic breast cancers that correlated with higher tumor grade, tumor size, aggressive basal subtype, and poor outcome in the first 10 years. See Dischinger PS et al., NF1 deficiency correlates with estrogen receptor signaling and diminished survival in breast cancer. NPJ Br Cancer. 2018;4:29. The loss of NF1 increases Ras effector activation, leading to increased extracellular signal-regulated kinase (ERK), v-akt murine thymoma viral oncogene homolog (AKT), and ribosomal protein S6 phosphorylation. See Kaul A et al., Akt- or MEK-mediated mTOR inhibition suppresses Nf1 optic glioma growth. Neuro Oncol. 2015;17(6):843-853.

Further, phosphatase and tensin homolog (PTEN) is a negative regulator of the PI3K pathway and mutations or loss of the PTEN gene contribute to cancer, poor disease outcomes, and germline Cowden's syndrome. Tumors with low PTEN or other cancer driver gene mutations such as phosphatidylinositol-4,5-bisphosphate 3-kinase, catalytic subunit alpha (PIK3CA) mutations are known to activate AKT. See Xing et al., Phase II trial of AKT inhibitor MK-2206 in patients with advanced breast cancer who have tumors with PIK3CA or AKT mutations, and/or PTEN loss/PTEN mutation. Breast Cancer Res. 2019;21(1):78. Although cancer patients with PIK3CA mutations, PTEN deletions (or mutations), or AKT mutations have been enrolled in several biomarker-driven studies, no significant correlation between these gene mutations and tumor response has been confirmed. See Janku et al., Targeting the PI3K pathway in cancer: are we making headway? Nature Rev Clin Oncol. 2018;15:273-91, 2018; Rodon et al., Development of PI3K inhibitors: lessons learned from early clinical trials. Nat Rev Clin Oncol. 2013;10:143-53.

Still further, as a member of the RAS oncogene family, the Kirsten rat sarcoma virus (KRAS) gene is the most frequently mutated oncogene. A KRAS mutation can lead to continuous activation of MAPK and PI3K pathways, contributing to the growth of cancer, and is associated with poor prognosis and resistance to therapy. See Haigis KM. KRAS alleles: the devil is in the detail. Trends Cancer, 2017;3(10):686-697; Zhuang R, et al., The prognostic value of KRAS mutation by cell-free DNA in cancer patients: A systematic review and meta-analysis. PLOS ONE. 2017;12(8):e0182562. In addition, aberrations in both the MAPK and PI3K pathways are known to coexist. As demonstrated in a study of patient tumors by Janku et al., KRAS mutations (38%) were found to coexist with mutant PIK3CA. See Janku F, et al., PIK3CA mutations frequently coexist with RAS and BRAF mutations in patients with advanced cancers. PLoS ONE. 2011;6(7):e22769. Therefore, even if tumors carry aberrations of PIK3CA, PTEN, or AKT that may predict efficacy of PI3K pathway inhibitors, the response to therapy targeting the PI3K pathway is likely to be limited in tumors bearing KRAS mutations.

Yet still further, metastatic hormone receptor positive (HR+) and human epidermal growth factor receptor 2 negative (HER2−) breast cancer is treated with endocrine therapy (ET). Unfortunately, ET resistance often develops, highlighting the need for treatment that can revert or delay resistance. See Vernieri C et al., Everolimus versus alpelisib in advanced hormone receptor-positive HER2-negative breast cancer: targeting different nodes of the PI3K/AKT/mTORC1 pathway with different clinical implications. Breast Cancer Res. 2020;22(1):33. In HR+/HER2− breast cancer, aberrant signaling of the PI3K pathway into enhanced activation leads to stimulation of the MAPK pathway and the estrogen receptor α (ERα) pathways. Specifically, ribosomal protein S6 kinase beta-1 (S6K1) and p90 ribosomal S6 kinase (RSK) synergistically regulate ERα transcription. RSK serves as a primer and S6K1 as maintenance for ERα Ser167 phosphorylation. Thus, activation of these pathways can induce activation of ERα and induce ET resistance. See Yamnik RL et al., mTOR/S6K1 and MAPK/RSK signaling pathways coordinately regulate estrogen receptor α serine 167 phosphorylation. FEBS Letters. 2010;584(1):124-128.

In view of the forgoing, there exists a need for new treatment methods in patients with advanced/metastatic cancers with dysregulation of the MAPK and/or PI3K pathway, and in particular, cancers (e.g., breast cancers) harboring NF1 aberrations, cancers with PTEN loss of gene or variants which lead to loss of function, cancers with oncogenic KRAS mutations, and HR+/HER2− breast cancers including those that have progressed on endocrine therapy.

### SUMMARY OF THE INVENTION

Accordingly, it is an object of the present disclosure to provide methods of treating a subject with a cancer having a dysregulated MAPK and/or PI3K pathway.

It is another object of the present disclosure to provide methods of treating a subject with a cancer, in particular a solid tumor, harboring an NF1 aberration (e.g., NF1 genetic variant).

It is another object of the present disclosure to provide methods of treating a cancer harboring a PTEN aberration (e.g., loss of gene or variants which lead to loss of function).

It is another object of the present disclosure to provide methods of treating a cancer with a KRAS mutation, such as a KRAS G12X mutation.

It is another object of the present disclosure to provide methods of treating HR+/HER2− breast cancers, such as those that have progressed on at least one prior therapy (e.g., endocrine therapy, cell cycle inhibitor therapy, etc.).

These and other objects, which will become apparent during the following detailed description, have been achieved by the inventors' discovery of 4-(4-(3-((2-(tert-butylamino)ethyl)amino)-6-(5-(trifluoromethyl)-1,3,4-oxadiazol-2-yl)pyridin-2-yl)piperidin-1-yl)-5,5-dimethyl-5H-pyrolo[2,3-d]pyrimidin-6(7H)-one (also known as “TAS0612”) or a pharmaceutically acceptable salt thereof, an orally available and highly potent triple kinase inhibitor of RSK, AKT, and S6K, with the potential for dual inhibition of aberrant MAPK and PI3K pathways, which has been discovered to be active in, and can be used to treat cancers such as those listed above. Thus, the present disclosure provides:
(1) A method of treating a subject with a cancer, e.g., a solid tumor, having an aberration in NF1, the method comprising administering to the subject an effective amount of 4-(4-(3-((2-(tert-butylamino)ethyl)amino)-6-(5-(trifluoromethyl)-1,3,4-oxadiazol-2-yl)pyridin-2-yl)piperidin-1-yl)-5,5-dimethyl-5H-pyrolo[2,3-d]pyrimidin-6(7H)-one (TAS0612) or a pharmaceutically acceptable salt thereof. This compound having the formula below is referred to as Compound (1):

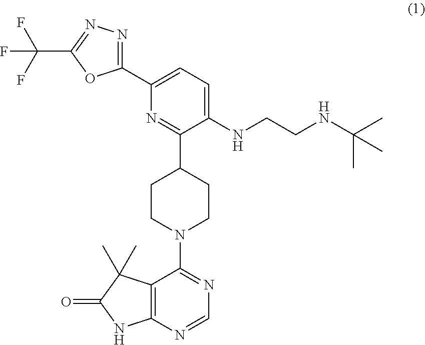 [SMILES: CC(C)(C)NCCNc1ccc(-c2nnc(C(F)(F)F)o2)nc1C1CCN(c2ncnc3c2C(C)(C)C(=O)N3)CC1]

(2) The method of (1), wherein the solid tumor is at least one selected from the group consisting of breast cancer, lung cancer, ovarian cancer, skin cancer, colon cancer, liver cancer, and esophagogastric cancer.
(3) The method of (1) or (2), wherein the solid tumor is breast cancer.
(4) The method of (3), wherein the breast cancer is human epidermal growth factor receptor 2 negative (HER2−) breast cancer.
(5) The method of any one of (1) to (4), wherein the subject is determined to have the aberration in NF1 prior to administering TAS0612 or a pharmaceutically acceptable salt thereof.
(6) The method of any one of (1) to (5), wherein the solid tumor has a co-occurring aberration in NF1 and at least one selected from the group consisting of KRAS, BRAF, PIK3CA, AKT1, and PTEN.
(7) The method of any one of (1) to (6), wherein the solid tumor harbors an inactivating NF1 genetic variant.
(8) The method of any one of (1) to (7), wherein TAS0612 or a pharmaceutically acceptable salt thereof is administered orally to the subject.
(9) The method of any one of (1) to (8), wherein TAS0612 or a pharmaceutically acceptable salt thereof is administered to the subject once per day (QD).
(10) The method of any one of (1) to (9), wherein from about 10 to about 960 mg of TAS0612 or a pharmaceutically acceptable salt thereof is administered to the subject per day.
(11) The method of any one of (1) to (10), wherein TAS0612 or a pharmaceutically acceptable salt thereof is administered daily to the subject for at least 28 days.
(12) A method of treating a subject with a hormone receptor positive and human epidermal growth factor receptor 2 negative (HR+/HER2−) breast cancer, the method comprising administering to the subject an effective amount of 4-(4-(3-((2-(tert-butylamino)ethyl)amino)-6-(5-(trifluoromethyl)-1,3,4-oxadiazol-2-yl)pyridin-2yl)piperidin-1-yl)-5,5-dimethyl-5H-pyrolo[2,3-d]pyrimidin-6(7H)-one (TAS0612) or a pharmaceutically acceptable salt thereof in combination with a second breast cancer therapy.
(13) The method of (12), wherein the second breast cancer therapy is at least one selected from the group consisting of endocrine therapy, cell cycle inhibitor therapy, and radiation therapy.
(14) The method of (12) or (13), wherein the second breast cancer therapy is endocrine therapy with tamoxifen and/or fulvestrant.
(15) The method of (12) or (13), wherein the second breast cancer therapy is cell cycle inhibitor therapy with abemaciclib, palbociclib, and/or ribociclib.
(16) The method of (12) or (13), wherein the second breast cancer therapy is radiation therapy.
(17) The method of any one of (12) to (16), wherein the HR+/HER2− breast cancer is a recurrent or refractory HR+/HER2− breast cancer.
(18) The method of (17), wherein the subject with the recurrent or refractory HR+/HER2− breast cancer has previously undergone a treatment regimen with an endocrine therapy and/or a cyclin-dependent kinase 4 and 6 (CDK4/6) inhibitor prior to administering TAS0612 or a pharmaceutically acceptable salt thereof.
(19) The method of (18), wherein the recurrent or refractory HR+/HER2− breast cancer acquired resistance to or intractability from the treatment regimen with the endocrine therapy and/or cyclin-dependent kinase 4 and 6 (CDK4/6) inhibitor.
(20) The method of any one of (17) to (19), wherein the recurrent or refractory HR+/HER2− breast cancer is endocrine therapy-resistant.
(21) The method of any one of (17) to (20), wherein the recurrent or refractory HR+/HER2− breast cancer is tamoxifen-resistant and/or fulvestrant-resistant.
(22) The method of any one of (17) to (21), wherein the recurrent or refractory HR+/HER2− breast cancer is CDK4/6 inhibitor-resistant.
(23) The method of any one of (12) to (22), wherein the HR+/HER2− breast cancer harbors an aberration in NF1.
(24) The method of any one of (12) to (23), wherein TAS0612 or a pharmaceutically acceptable salt thereof is administered orally to the subject.
(25) The method of any one of (12) to (24), wherein TAS0612 or a pharmaceutically acceptable salt thereof is administered to the subject once per day (QD).
(26) The method of any one of (12) to (25), wherein from about 10 to about 960 mg of TAS0612 or a pharmaceutically acceptable salt thereof is administered to the subject per day.
(27) The method of any one of (12) to (26), wherein TAS0612 or a pharmaceutically acceptable salt thereof is administered daily to the subject for at least 28 days.
(28) A method of treating a subject with a cancer having an aberration in PTEN, the method comprising administering to the subject an effective amount of 4-(4-(3-((2-(tert-butylamino)ethyl)amino)-6-(5-(trifluoromethyl)-1,3,4-oxadiazol-2-yl) pyridin-2-yl)piperidin-1-yl)-5,5-dimethyl-5H-pyrolo[2,3-d]pyrimidin-6(7H)-one (TAS0612) or a pharmaceutically acceptable salt thereof.
(29) The method of (28), wherein the cancer is at least one selected from the group consisting of breast cancer, thyroid cancer, renal cell carcinoma, endometrial cancer, colorectal cancer, melanoma, glioblastoma, prostate cancer, ovarian cancer, and lung cancer.
(30) The method of (28) or (29), wherein the cancer is endometrial cancer.
(31) The method of any one of (28) to (30), wherein the subject is determined to have the aberration in PTEN prior to administering TAS0612 or a pharmaceutically acceptable salt thereof.
(32) The method of any one of (28) to (31), wherein the cancer has a co-occurring aberration in PTEN and at least one selected from the group consisting of KRAS, BRAF, PIK3CA, AKT1, EGFR, HER2, TP53, NF1, and BRCA.
(33) The method of any one of (28) to (32), wherein the cancer has a co-occurring aberration in PTEN and PIK3CA.
(34) The method of any one of (28) to (33), wherein the aberration in PTEN is PTEN loss of gene or variants which lead to loss of function.
(35) The method of any one of (28) to (34), wherein TAS0612 or a pharmaceutically acceptable salt thereof is administered orally to the subject.
(36) The method of any one of (28) to (35), wherein TAS0612 or a pharmaceutically acceptable salt thereof is administered to the subject once per day (QD).
(37) The method of any one of (28) to (36), wherein from about 10 to about 960 mg of TAS0612 or a pharmaceutically acceptable salt thereof is administered to the subject per day.
(38) The method of any one of (28) to (37), wherein TAS0612 or a pharmaceutically acceptable salt thereof is administered daily to the subject for at least 28 days.
(39) A method of treating a subject with a cancer having an aberration in KRAS, the method comprising administering to the subject an effective amount of 4-(4-(3-((2-(tert-butylamino)ethyl)amino)-6-(5-(trifluoromethyl)-1,3,4-oxadiazol-2-yl)pyridin-2-yl)piperidin-1-yl)-5,5-dimethyl-5H-pyrolo[2,3-d]pyrimidin-6(7H)-one (TAS0612) or a pharmaceutically acceptable salt thereof.
(40) The method of (39), wherein the cancer is at least one selected from the group consisting of colorectal cancer, lung cancer, pancreatic cancer, endometrial cancer, skin cancer, ovarian cancer, biliary cancer, and breast cancer.
(41) The method of (39) or (40), wherein the subject is determined to have the aberration in KRAS prior to administering TAS0612 or a pharmaceutically acceptable salt thereof.
(42) The method of any one of (39) to (41), wherein the cancer has a co-occurring aberration in KRAS and at least one selected from the group consisting of BRAF, PIK3CA, PTEN, EGFR, TP53, BRCA, APC, MTOR, and SMAD4.
(43) The method of any one of (39) to (42), wherein the cancer has a co-occurring aberration in KRAS and at least one selected from the group consisting of PIK3CA and PTEN.
(44) The method of any one of (39) to (43), wherein the cancer harbors a KRAS G12C mutation or a KRAS G12D mutation.
(45) The method of (44), wherein the subject is administered TAS0612 or a pharmaceutically acceptable salt thereof in combination with a KRAS G12C-specific inhibitor or a KRAS G12D-specific inhibitor.
(46) The method of any one of (39) to (45), wherein TAS0612 or a pharmaceutically acceptable salt thereof is administered orally to the subject.
(47) The method of any one of (39) to (46), wherein TAS0612 or a pharmaceutically acceptable salt thereof is administered to the subject once per day (QD).
(48) The method of any one of (39) to (47), wherein from about 10 to about 960 mg of TAS0612 or a pharmaceutically acceptable salt thereof is administered to the subject per day.
(49) The method of any one of (39) to (48), wherein TAS0612 or a pharmaceutically acceptable salt thereof is administered daily to the subject for at least 28 days.
(50) An antitumor agent for treating a subject with a cancer having a dysregulated MAPK and/or PI3K pathway, the antitumor agent comprising 4-(4-(3-((2-(tert-butylamino)ethyl)amino)-6-(5-(trifluoromethyl)-1,3,4-oxadiazol-2-yl)pyridin-2-yl) piperidin-1-yl)-5,5-dimethyl-5H-pyrolo[2,3-d]pyrimidin-6(7H)-one (TAS0612) or a pharmaceutically acceptable salt thereof.
(51) Use of 4-(4-(3-((2-(tert-butylamino)ethyl)amino)-6-(5-(trifluoromethyl)-1,3,4-oxadiazol-2-yl)pyridin-2-yl)piperidin-1-yl)-5,5-dimethyl-5H-pyrolo[2,3-d]pyrimidin-6(7H)-one (TAS0612) or a pharmaceutically acceptable salt thereof in the treating of a subject with a cancer having a dysregulated MAPK and/or PI3K pathway.
(52) 4-(4-(3-((2-(tert-butylamino)ethyl)amino)-6-(5-(trifluoromethyl)-1,3,4-oxadiazol-2-yl)pyridin-2-yl)piperidin-1-yl)-5,5-dimethyl-5H-pyrolo[2,3-d]pyrimidin-6(7H)-one (TAS0612) or a pharmaceutically acceptable salt thereof for use in treating a subject with a cancer having a dysregulated MAPK and/or PI3K pathway.

### BRIEF DESCRIPTION OF THE DRAWINGS

The foregoing paragraphs have been provided by way of general introduction, and are not intended to limit the scope of the following claims. The described embodiments, together with further advantages, will be best understood by reference to the following detailed description when considered in conjunction with the accompanying drawing, wherein:

FIG. 1A illustrates the effect of NF1 depletion by gene silencing on the MAPK and PI3K signal transduction cascades; NF1 depletion in the estrogen receptor alpha (ERα) positive and HER2 negative breast cancer derived MCF7 cells exhibited activation of both MAPK and PI3K signaling under hormone depleted culture condition; data are shown as immunoblots detecting NF1, pAKT, pERK, pS6, and ERα proteins in control and NF1 siRNA-transfected MCF7 cells, and FIG. 1B is a schema indicating changes in intracellular signaling upon depletion of NF1.

FIGS. 2A-2B illustrate the effect of Compound (1), MEK inhibitor Trametinib, PI3K inhibitor Alpelisib, and ERα-targeted degrader fulvestrant on the signaling and apoptosis-inducing potential in the NF1 depleted MCF7 (FIG. 2A) and T47D cells (FIG. 2B); data are shown as immunoblots detecting NF1, pPRAS40, pYB1, pS6, ERα, and cleaved PARP.

FIG. 3 illustrates that Compound (1) induces target inhibition and apoptosis in ER+/HER2− breast cancer cell lines, regardless of NF1 or estrogen status, and the effect is further enhanced when combined with fulvestrant; data are shown as immunoblots detecting NF1, pPRAS40, pYB1, pS6, ERα, and cleaved PARP.

FIGS. 4A-4D illustrate the effect of Compound (1) against PTEN-deficient cell growth and apoptosis induction; FIG. 4A shows that Compound (1) exhibited potent growth inhibition in small cancer cell panel where the IC50 values were apparently associated with the PTEN gene alterations; FIG. 4B shows the results of confirmatory large cell panel analysis; FIG. 4C shows that Compound (1) induced marked apoptosis in a PTEN-mutated HEC-6 cancer cell line in a dose-dependent manner; FIG. 4D shows that Compound (1) induced marked apoptosis in a PTEN-mutated MFE-319 cancer cell line in a dose-dependent manner.

FIGS. 5A-5B illustrates the antitumor effect of Compound (1) or sotorasib or the combination of Compound (1) and sotorasib in nude mice bearing the KRAS_G12C and PIK3CA_K111E mutated SW1573_human lung tumor xenografts, as a function of tumor volume (TV) (FIG. 5A) and body weight change (BWC) (FIG. 5B); results are shown as the mean±standard error in both TV and BWC (n=5 animals/group).

FIGS. 6A-6B illustrates the antitumor effect of Compound (1) or sotorasib or the combination of Compound (1) and sotorasib in nude mice bearing the KRAS_G12C mutated LU65_human lung tumor xenografts, as a function of TV (FIG. 6A) and BWC (FIG. 6B); results are shown as the meantstandard error in both TV and BWC (n=5 animals/group).

FIGS. 7A-7B illustrates the antitumor effect of Compound (1) or trametinib in nude mice bearing LS180_human colon tumor xenografts carrying mutations of KRAS_G12D, PIK3CA_H1047R, and PTEN_167K, as a function of TV (FIG. 7A) and BWC (FIG. 7B); results are shown as the mean±standard error in both TV and BWC (n=5 animals/group).

FIGS. 8A-8B illustrates the antitumor effect of Compound (1) or trametinib or the combination of Compound (1) and trametinib in nude mice bearing KRAS_G12D mutated AsPC-1_human pancreas tumor xenografts, as a function of TV (FIG. 8A) and BWC (FIG. 8B); results are shown as the meantstandard error in both TV and BWC (n=5 animals/group).

### DETAILED DESCRIPTION OF THE PREFERRED EMBODIMENTS

Compound (1) is a novel, selective, potent, and orally available multikinase inhibitor with in vitro mean half-maximal inhibitory concentration (IC₅₀) activity against all target kinase isoforms (RSK1, RSK2, RSK3, RSK4, AKT1, AKT2, AKT3, S6K1, and S6K2) ranging from 0.16 to 1.7 nmol/L. For this reason, Compound (1) may function as a dual inhibitor of both the MAPK and PI3K pathways, provide an anticancer effect against cancers having a dysregulated MAPK and/or PI3K pathway, and overcome/reverse resistance to other anticancer agents such as anti-hormonal agents and targeted therapy agents (e.g., tyrosine kinase inhibitors). Compound (1) is described in U.S. Pat. No. 10,538,528 and its corresponding WO2017/200087 (see Example 32), the contents of which are incorporated herein by reference in their entirety.

Compound (1) can be used directly (free form) or in the form of a pharmaceutically acceptable salt. The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. The pharmaceutically acceptable salt of Compound (1) is not particularly limited. Examples of such salts include base addition salts, and acid addition salts. Examples thereof include addition salts with inorganic acids such as hydrochloric acid, hydrobromic acid, sulfuric acid, sulfamic acid, phosphoric acid, nitric acid, perchloric acid, and the like; organic acids such as acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid (tosylic acid), methanesulfonic acid (mesylic acid), ethane disulfonic acid (esylic acid), oxalic acid, isethionic acid, formic acid, and the like; salts with alkali metals such as potassium, sodium, and the like; salts with alkaline earth metals such as calcium, magnesium, and the like; and salts with organic bases such as ammonium salts, ethylamine salts, alginate, organic amine salts, such as trimethylamine salts, triethylamine salts, dicyclohexylamine salts, ethanolamine salts, diethanolamine salts, triethanolamine salts, procaine salts, and N,N′-dibenzylethylenediamine salts, and the like. The pharmaceutically acceptable salts can be synthesized by conventional chemical methods, generally by reacting Compound (1) with a stoichiometric amount or sub-stoichiometric amount (e.g., 0.5 eq) of the appropriate base or acid in water or in an organic solvent (e.g., ether, ethyl acetate, ethanol, isopropanol, or acetonitrile), or in a mixture of the two.

Compound (1) or a pharmaceutically acceptable salt thereof may be in the form of a “solvate”, which refers to a physical association of a referenced compound with one or more solvent molecules, whether organic or inorganic. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. Solvate encompasses both solution phase and isolable solvates. Exemplary solvent molecules which may form the solvate include, but are not limited to, water, methanol, ethanol, n-propanol, isopropanol, n-butanol, isobutanol, tert-butanol, ethyl acetate, glycerin, acetone, and the like.

Compound (1) or pharmaceutically acceptable salt thereof may exist in an amorphous form or crystalline form. Compounds of a single crystalline form or a mixture of many crystalline forms or in a co-crystal form with the other components are within the scope of the present disclosure. Crystals can be produced with the application of known crystallization techniques. Preferred crystal forms are those having good stability, excellent oral absorbability, high chemical purity, are non-hygroscopic, and are suitable for mass production.

Exemplary methods which can be used for synthesizing Compound (1) are described in U.S. Pat. No. 10,538,528 and its corresponding WO2017/200087 (see Example 32), the contents of which are incorporated herein by reference in its entirety.

The terms “treat”, “treating”, or the “treatment” of cancers in the present disclosure includes any effect, e.g., lessening, reducing, modulating, stabilizing, ameliorating or eliminating, that results in the improvement of the condition, disease, disorder, and the like, or ameliorating a symptom thereof. Specifically, these terms may refer to: (1) a stabilization, reduction (e.g., by more than 10%, 20%, 30%, 40%, 50%, preferably by more than 60% of the population of cancer cells and/or tumor size as compared to prior to administration), or elimination of the cancer cells, (2) inhibiting cancerous cell division and/or cancerous cell proliferation, (3) relieving to some extent (or, preferably, eliminating) one or more symptoms associated with a pathology related to or caused in part by unregulated or aberrant cellular division, (4) an increase in disease-free, relapse-free, progression-free, and/or overall survival, duration, or rate, (5) a decrease in hospitalization rate, (6) a decrease in hospitalization length, (7) eradication, removal, or control of primary, regional and/or metastatic cancer, (8) a stabilization or reduction (e.g., by at least 10%, 20%, 30%, 40%, 50%, 60%, 70%, preferably at least 80% relative to the initial growth rate) in the growth of a tumor or neoplasm, (9) an impairment in the formation of a tumor, (10) a reduction in mortality, (11) an increase in the response rate, the durability of response, or number of patients who respond or are in remission, (12) the size of the tumor is maintained and does not increase or increases by less than 10%, preferably less than 5%, preferably less than 4%, preferably less than 2%, (13) a decrease in the need for surgery (e.g., colectomy, mastectomy), and/or (14) preventing or reducing the metastasis of cancer cells.

The cancers which can be treated herein are those that are sensitive to inhibition of the MAPK and/or PI3K pathways. In particular, the cancers which can be treated herein are those in which the Ras/Raf/MEK/ERK/RSK and/or Ras/PI3K/PTEN/AKT/mTOR/S6K cascades are aberrantly activated.

Examples of types of cancers which can be treated herein include, but are not limited to, solid tumors such as glandular tumors, carcinoid tumors, undifferentiated carcinomas, angiosarcoma, adenocarcinoma, gastrointestinal cancers (e.g., colorectal cancers (“CRC”) including colon cancer, rectal cancer, and cecal cancer, biliary cancers including gall bladder cancer and bile duct cancer (cholangiocarcinoma), anal cancer, esophageal cancer, gastric (stomach) cancer, gastrointestinal carcinoid tumor(s), gastrointestinal stromal tumor(s) (“GIST”), liver cancer, duodenal cancer, appendiceal cancer, and small intestine cancer), lung cancers (e.g., non-small cell lung cancer (“NSCLC”), squamous-cell lung carcinoma, large-cell lung carcinoma, small cell lung carcinoma, invasive mucinous adenocarcinoma, mesothelioma and other lung cancers such as bronchial tumors and pleuropulmonary blastoma), urological cancers (e.g., kidney (renal) cancer, transitional cell cancer (“TCC”) of kidney, TCC of the renal pelvis and ureter (“PDQ”), bladder cancer, urethral cancer and prostate cancer), head and neck cancers (e.g., eye cancer, retinoblastoma, intraocular melanoma, hypopharyngeal cancer, pharyngeal cancer, laryngeal cancer, laryngeal papillomatosis, metastatic squamous neck cancer with occult primary, sinonasal squamous cell carcinoma (SNSCC), oral (mouth) cancer, lip cancer, throat cancer, oropharyngeal cancer, esthesioneuroblastoma, nasal cavity and paranasal sinus cancer, nasopharyngeal cancer, and salivary gland cancer), endocrine cancers (e.g., thyroid cancer, parathyroid cancer, multiple endocrine neoplasia syndromes, thymoma and thymic carcinoma, pancreatic cancers including pancreatic ductal adenocarcinoma (“PDAC”), pancreatic neuroendocrine tumors and islet cell tumors), breast cancers (extrahepatic ductal carcinoma in situ (“DCIS”), lobular carcinoma in situ (“LCIS”), triple negative breast cancer, and inflammatory breast cancer), male and female reproductive cancers (e.g., cervical cancer, ovarian cancer, endometrial cancer, uterine sarcoma, uterine cancer, vaginal cancer, vulvar cancer, gestational trophoblastic tumor (“GTD”), extragonadal germ cell tumor, extracranial germ cell tumor, germ cell tumor, testicular cancer and penile cancer), brain and nervous system cancers (e.g., astrocytomas, brain stem glioma, brain tumor, glioblastoma (GBM), craniopharyngioma, central nervous system (“CNS”) cancer, chordomas, ependymoma, embryonal tumors, neuroblastoma, paraganglioma, atypical teratoid, oligodendroma, oligodendroastrocytoma, oligodendroglioma, anaplastic oligodendroastrocytoma, ganglioglioma, central neurocytoma, medulloblastoma, germinoma, meningioma, neurilemmoma, GH secreting pituitary adenoma, PRL-secreting pituitary adenoma, ACTH-secreting pituitary adenoma, nonfunctional pituitary adenoma, hemangioblastoma, and epidermoid tumor), skin cancers (e.g., basal cell carcinoma (“BCC”), squamous cell skin carcinoma (“SCC”), Merkel cell carcinoma and melanoma), tissue and bone cancers (e.g., soft-tissue sarcoma, rhabdomyosarcoma, fibrous histiocytoma of bone, Ewing sarcoma, malignant fibrous histiocytoma of bone (“MFH”), osteosarcoma and chondrosarcoma), cardiovascular cancers (e.g., heart cancer and cardiac tumors), appendix cancers, childhood and adolescent cancers (e.g., adrenocortical carcinoma childhood, midline tract carcinoma, hepatocellular carcinoma (“HCC”), hepatoblastoma and Wilms' tumor) and viral-induced cancers (e.g., HHV-8 related cancers (Kaposi sarcoma) and HIV/AIDS related cancers).

Cancers also suitable for treatment may include, but are not limited to, hematological and plasma cell malignancies (e.g., cancers that affect blood, bone marrow and/or lymph nodes) such as multiple myeloma, leukemias and lymphomas, myelodysplastic syndromes and myeloproliferative disorders. Leukemias include, without limitation, acute lymphoblastic leukemia (“ALL”), acute myelogenous (myeloid) leukemia (“AML”), chronic lymphocytic leukemia (“CLL”), chronic myelogenous leukemia (“CML”), acute monocytic leukemia (“AMoL”), hairy cell leukemia, and/or other leukemias. Lymphomas include, without limitation, Hodgkin's lymphoma and non-Hodgkin's lymphoma (“NHL”). In some embodiments, NHL is B-cell lymphomas and/or T-cell lymphomas. In some embodiments, NHL includes, without limitation, diffuse large B-cell lymphoma (“DLBCL”), small lymphocytic lymphoma (“SLL”), chronic lymphocytic leukemia (“CLL”), mantle cell lymphoma (“MCL”), Burkitt's lymphoma, cutaneous T-cell lymphoma including mycosis fungoides and Sézary syndrome, AIDS-related lymphoma, follicular lymphoma, lymphoplasmacytic lymphoma (Waldenstrom's macroglobulinemia (“WM”)), primary central nervous system (CNS) lymphoma, central nervous system malignant lymphoma, and/or other lymphomas.

The treatment methods of the present disclosure are particularly useful in the treatment of solid tumors/cancers such as lung cancer (e.g., non-small cell lung cancer (“NSCLC”), invasive mucinous adenocarcinoma, etc.), breast cancer (e.g., extrahepatic ductal carcinoma in situ (“DCIS”), lobular carcinoma in situ (“LCIS”), etc.), male and female reproductive cancers (e.g., endometrial/uterine cancer, ovarian cancer, etc.), urological cancers (e.g., prostate cancer, etc.), gastrointestinal cancer (e.g., colon/colorectal cancer, cecum, etc.), endocrine cancer (e.g., pancreatic cancer, etc.), and skin cancers (e.g., melanoma).

The methods disclosed herein may also be used as a tumor-agnostic treatment for malignancies which are sensitive to inhibition of the MAPK and/or PI3K pathways.

While cancers at various stages and resectabilities may respond to the disclosed treatment, the methods herein may be particularly useful in the treatment of advanced (stage III) and metastatic (stage IV) disease, “recurrent,” and “refractory” cancers—cancer that heretofore has failed to respond to medical treatment. “Recurrent” cancers are cancers that have recurred (come back), usually after a period of time during which the cancer could not be detected. The cancer may come back to the same place as the original (primary) tumor or to another place in the body. “Refractory” cancers may present as resistance/intractability from the start, or the acquisition of resistance/intractability by the cancer cells during the course of prior therapy, and thus can include relapsed cancer that responds initially to treatment, but returns, often in a more aggressive/resistant form. The recurrent or refractory cancer may be either resectable or unresectable.

Cancers responsive to treatment in the present disclosure may in some cases harbor an aberration in one or more cancer driver genes/component(s) associated with the MAPK and/or PI3K pathways (e.g., NF1, PTEN, PIK3CA, KRAS, etc.) that results in or contributes to cancer formation and/or development.

“Cancer driver genes” are genes that give cells a growth advantage when they are genetically altered, helping tumors proliferate. Cancer driver genes generally fall into two classes: tumor suppressor genes and oncogenes. “Tumor suppressor genes”, or anti-oncogenes, provide negative control of cell proliferation. Loss-of-function of the proteins encoded by these genes, through deletion, mutation, or inactivation of the gene, liberates the cell from growth constraints and contributes to malignant transformation. “Oncogenes” are genes that normally help cells grow, that when genetically altered result in activated or over-expressed levels of proteins that can cause those cells designated for apoptosis to survive and proliferate instead. Thus, the gain-of-function of oncogenes together with the loss-of-function of tumor suppressor genes determine the processes that control tumor formation and development.

In the present disclosure, a genetic/protein “aberration” includes protein overexpression, gene amplification (e.g., copy-number alterations), gene/protein mutations (e.g., insertion, substitution, or deletion mutations, including those categorized as nonsense, missense, splice, or frameshift mutations), chromosomal translocation/insertion/inversion, gene rearrangement or gene fusion (a subset of gene rearrangements), promoter hypermethylation, post-translational modifications, and the like, including combinations thereof, that result in or contributes to cancer formation and/or development.

The cancers, e.g., solid tumors, treated herein may harbor an NF1 aberration. NF1 is a tumor suppressor gene that encodes for neurofibromin protein, which acts as a repressor of RAS-GTP activation, with loss of NF1 resulting in RAS activation and downstream to the MAPK pathway activation. Preferred cancers harboring an NF1 aberration are solid tumors.

Aberrations in NF1 may be in the form of one or more inactivating mutations, including, but not limited to, nonsense, frameshift, missense, splice, or deletion mutations. The inactivating mutation may be a germline mutation, which are known to increase the risk of breast cancer in women under 50 years old that could lead to an increase of cancer-related death, or a somatic mutation, which are rare in primary cancer, but are associated with poor prognosis and an increased risk of recurrence. See Madanikia SA, et al., Increased risk of breast cancer in women with NF1. Am J Med Genet A 2012;158A:3056-60; Uusitalo E, et al., Distinctive cancer associations in patients with neurofibromatosis type 1. J Clin Oncol 2016; 34:1978-86; Sharif S, et al., Women with neurofibromatosis 1 are at a moderately increased risk of developing breast cancer and should be considered for early screening. J Med Genet 2007; 44:481-4; and Griffith OL, et al., The prognostic effects of somatic mutations in ER-positive breast cancer. Nat Commun 2018; 9:3476.

A preferred embodiment of the present disclosure involves treating a subject with a solid tumor, particularly a breast cancer, e.g., advanced/metastatic HER2− breast cancer, harboring one or more NF1 genetic variants. “HER2−” means that HER2 expression levels are considered to be within normal limits, e.g., compared to healthy cells. Such HER2− breast cancer includes ER/PR(+), HER2− breast cancer and triple negative breast cancer (TNBC).

NF1 genetic variants, which can be interchangeably referred to as NF1 mutations, may include, but are not limited to, R1204W, X465_splice, S2751Rfs*27?, 1679Dfs*21, T467Hfs*3, P678Rfs*10, P388T, Y628Tfs*3, V2205A, N2341Tfs*5, Q1336*, and N184Wfs*17. Other examples of NF1 genetic variants may include, but are not limited to, Q83Ter, R192Ter, R304Ter, P504fs, G629R, T770fs, Q948Ter, c.3198-1G>C, S1078Ter, A1098fs, S1329Ter, K1429Ter, Q1515K, K1752N, c.5268+1G>T, S2435del, and G2683A. See Pearson, A., et al., Inactivating NF1 Mutations Are Enriched in Advanced Breast Cancer and Contribute to Endocrine Therapy Resistance. Clin Cancer Res, 2020; (26)(3), 608-622. Unless specified otherwise, any reference to NF1 amino acid sequence information is based on human wild-type NF1 isoform 1, which is accessible from the National Center for Biotechnology Information (NCBI) Protein Database as Accession No. NP_001035957, etc. Isoforms of NF1 are also known by those of ordinary skill in the art, and the present disclosure also encompasses those isoforms. With regard to alterations (e.g., mutations) in NF1 discussed herein, it should be understood that the alteration in the isoform may be located in a different position from the position identified for NF1 due to deletion or insertion of an amino acid(s) in the isoform, but that the alteration in the isoform nevertheless corresponds to the position identified for NF1.

Besides breast cancer, other specific examples of cancer types having an NF1 aberration that may be treated herein include, but are not limited to, lung cancer (e.g., NSCLC), ovarian cancer, brain and nervous system cancers, skin cancer (e.g., melanoma), and gastrointestinal cancers such as colon cancer, liver cancer, and esophagogastric cancer.

The cancers harboring an NF1 aberration may in some cases also harbor a co-occurring aberration in one or more other cancer driver genes, e.g., those associated with the MAPK and/or PI3K pathway, including, but not limited to, Ras (e.g., HRAS, KRAS, NRAS), v-Raf murine sarcoma viral oncogene homolog B (BRAF) (e.g., G464V and/or V600E, etc.), PIK3CA, AKT1 (e.g., E17K and/or L52R, etc.), PTEN, estrogen receptor 1 (ESR1), and tumor protein p53 gene (TP53).

With respect to PIK3CA aberrations, particular mention is made to one or more PIK3CA genetic variants, which can be interchangeably referred as PIK3CA mutations, examples of which include, but are not limited to, K111E, E542X including E542K/A/G/Q/D, E545X including E545K/A/G/Q/D, L866F, K567R, and H1047X including H1047R/L/Y/Q. Unless specified otherwise, any reference to PIK3CA amino acid sequence information is based on human wild-type PIK3CA isoform α, which is accessible from the National Center for Biotechnology Information (NCBI) Protein Database as Accession No. NP_006209.2, etc. Isoforms of PIK3CA are also known by those of ordinary skill in the art, and the present disclosure also encompasses those isoforms. It should be understood that the alteration in isoforms may be located in a different position from the position identified for PIK3CA due to deletion or insertion of an amino acid(s) in the isoform, but that the alteration in the isoform nevertheless corresponds to the position identified for PIK3CA.

The cancers treated herein may harbor a PTEN aberration. PTEN encodes a ubiquitously expressed phosphatase that counteracts the PI3K/AKT/mTOR cascade. PTEN loss-of-function can cause a spectrum of phenotypes including benign overgrowths, malignancies, and metabolic and neurodevelopmental disorders. Germline PTEN aberrations are associated with elevated lifetime risks of breast cancer (estimated lifetime risk of 85%), thyroid cancer (35%), renal cell carcinoma (34%), endometrial cancer (28%), colorectal cancer (9%), and melanoma (6%). See Tan MH, et al., Lifetime cancer risks in individuals with germline PTEN mutations. Clin Cancer Res. 2012;18(2):400-407. Moreover, individuals with germline PTEN mutations have a 7-fold increased risk of developing a second primary malignant neoplasm compared to the general population in the United States.

Aberrations in PTEN leading to loss-of-function/PTEN-deficiency may be in the form of one or more inactivating mutations, including, but not limited to, nonsense, frameshift, missense, and deletion mutations, chromosomal deletions, promoter hypermethylations, and post-translational modifications. The cancers with a PTEN aberration may express truncated protein, full length protein, or complete loss of protein, which can be caused by a germline mutation or a somatic mutation.

Examples of PTEN loss of gene or variants which lead to loss of function, which can be interchangeably referred as PTEN mutation, include, but are not limited to, 167K, K267X including K267Rfs*9/*/fs*, R234Afs*1, L247*, C71Y, L112P, C124S, R130X including R130Q/G/L/P/*/fs*, C136Y, Y155C, Q214*, R233*, Q245*, E299*, T319X including T319*/fs*, N323fs* and R335 *. See Sun, Y., et al. PTENα functions as an immune suppressor and promotes immune resistance in PTEN-mutant cancer. Nat Commun 12, 5147 (2021). Unless specified otherwise, any reference to PTEN amino acid sequence information is based on the human wild-type PTEN isoform which is accessible from the National Center for Biotechnology Information (NCBI) Protein Database as Accession No. NP_000305.3, etc. Isoforms of PTEN are also known by those of ordinary skill in the art, and the present disclosure also encompasses those isoforms. It should be understood that the alteration in isoforms may be located in a different position from the position identified for PTEN due to deletion or insertion of an amino acid(s) in the isoform, but that the alteration in the isoform nevertheless corresponds to the position identified for PTEN.

A preferred embodiment of the present disclosure involves treating a subject with advanced/metastatic cancers, particularly solid tumors, with one or more PTEN aberrations, such as PTEN loss of gene or variants which lead to loss of function. Specific examples of such types of cancers include, but are not limited to, breast cancer, thyroid cancer, renal cell carcinoma, endometrial cancer, colorectal/cecal cancer, melanoma, glioblastoma (brain tumor), prostate cancer, ovarian cancer, and lung cancer.

In addition to a PTEN aberration, the cancer may in some cases also have a co-occurring aberration in one or more other cancer driver genes, e.g., those associated with the MAPK and/or PI3K pathway, including, but not limited to Ras (e.g., HRAS, KRAS, NRAS), (BRAF) (e.g., G464V and/or V600E, etc.), PIK3CA (e.g., those mutants described previously), AKT1 (e.g., E17K and/or L52R, etc.), epidermal growth factor receptor (EGFR), HER2, TP53, NF1, and breast cancer gene (BRCA). In particular, cancers harboring a co-occurring aberration in both PTEN and PIK3CA have been identified as highly sensitive to Compound (1) and these cancers thus represent preferred targets for treatment herein.

The cancers treated herein may harbor a KRAS aberration, in particular, a KRAS mutation. KRAS is a proto-oncogene located at a critical signaling junction between extracellular growth receptors and pro-growth pathways, that when mutated, hyperactivates many downstream effector pathways, such as MAPK and/or PI3K signaling pathways. KRAS is one of the most frequently mutated genes in cancer, with the highest frequencies in colorectal adenocarcinoma, lung adenocarcinoma, multiple myeloma, and pancreatic adenocarcinoma. The genetic interactions of oncogenic KRAS mutations are allele- and tissue-specific, leading to inconsistent therapeutic responses and clinical outcomes. See Cook, J.H. et al., The origins and genetic interactions of KRAS mutations are allele- and tissue-specific. Nat Commun 12, 1808 (2021). The gain-of-function KRAS aberration may be a germline mutation or a somatic mutation.

The KRAS aberration may occur, e.g., in codons 12, 13, 61, and 146, with specific mention being made to activating mutations (e.g., missense) in codon 12. KRAS genetic variants, which can be interchangeably referred as KRAS mutations, may include, but are not limited to, one or more point mutations such as G12X (where X is e.g., A, C, D, R, S or V), G13X (where X is e.g., A, C, D, R, S or V), Q61X (where X is e.g., E, H, K, L, P, and R), and A146X (where X is e.g., E, G, P, S, T, and V). In particular, the cancer treated herein is a KRAS G12X-mutant cancer, preferably a KRAS G12C-mutant cancer or a KRAS G12D-mutant cancer. Unless specified otherwise, any reference to KRAS amino acid sequence information is based on human wild-type KRAS isoform a, which is accessible from the National Center for Biotechnology Information (NCBI) Protein Database as Accession No. NP_001356715.1, etc. Isoforms of KRAS are also known by those of ordinary skill in the art, and the present disclosure also encompasses those isoforms. With regard to alterations (e.g., mutations) in KRAS discussed herein, it should be understood that the alteration in the isoform may be located in a different position from the position identified for KRAS due to deletion or insertion of an amino acid(s) in the isoform, but that the alteration in the isoform nevertheless corresponds to the position identified for KRAS.

A preferred embodiment of the present disclosure involves treating a subject with advanced/metastatic cancers, particularly solid tumors, with KRAS G12C mutations. Another preferred embodiment of the present disclosure involves treating a subject with advanced/metastatic cancers, particularly solid tumors, with KRAS G12D mutations. Specific examples of cancers types known to have such mutational signatures, and are candidates for treatment herein, include, but are not limited to, colorectal cancer, lung cancer (e.g., NSCLC), pancreatic cancer, endometrial cancer, skin cancer, ovarian cancer, biliary cancer, and breast cancer.

In addition to a KRAS aberration, the cancer may in some cases also have a co-occurring aberration in one or more other cancer driver genes, e.g., those associated with the MAPK and/or PI3K pathway, including, but not limited to BRAF (e.g., G464V and/or V600E), PIK3CA (e.g., those described previously), PTEN (e.g., those described previously), AKT1 (e.g., E17K and/or L52R, etc.), EGFR, TP53, BRCA, adenomatous polyposis coli (APC), mechanistic target of rapamycin (MTOR), and mothers against decapentaplegic homolog 4 (SMAD4). In particular, KRAS mutant-cancers (e.g., G12C or G12D mutants) having a co-occurring aberration in PIK3CA, PTEN, or both PIK3CA and PTEN have been found to be markedly sensitive to Compound (1) and these cancers thus represent preferred targets for treatment herein.

The subject with cancer harboring a KRAS aberration may be treated with Compound (1) or its pharmaceutically acceptable salt as a stand-alone therapy. Alternatively, the subject with cancer harboring a KRAS aberration may be treated with Compound (1) or its pharmaceutically acceptable salt in combination with a KRAS mutant-specific inhibitor. The KRAS mutant-specific inhibitor may include inhibitors which target and bind to specific KRAS mutant protein, or other downstream pathway inhibitors active against certain oncogenic KRAS-mutant cancers (i.e., those that block or attenuate the action of certain KRAS mutant proteins without specifically binding to the KRAS protein). Such combination therapy is intended to cover both simultaneously administered and sequentially (as pre- or post-treatment) administered combination therapy.

For example, cancers harboring a KRAS G12C mutation may be treated with Compound (1) or its pharmaceutically acceptable salt in combination with a KRAS G12C-specific inhibitor. Examples of KRAS G12C-specific inhibitors include, but are not limited to, AMG510 (sotorasib), MRTX849 (adagrasib), ARS-1620, ARS-853, JNJ-74699157 (ARS-3248), LY3499446, GDC-6036, D-1553, JDQ433, JAB-21822, and RM-007.

In another example, cancers harboring a KRAS G12D mutation may be treated with Compound (1) or its pharmaceutically acceptable salt in combination with a KRAS G12D-specific inhibitor. Examples of KRAS G12D-specific inhibitors include, but are not limited to, MRTX1133 and KRpep-2d.

The cancer treated herein may be hormone receptor positive (HR+) breast cancer, preferably a HR+and HER2− breast cancer. “HR+” means a cancer in which estrogen receptors, most notably ERα, are present/detectable allowing the cancer to use estrogen to grow (ER+), a cancer in which progesterone receptors are present/detectable allowing the cancer to use progesterone to grow (PR+), or a cancer which is both ER+and PR+.

The subject with HR+/HER2− breast cancer may be treated with Compound (1) or its pharmaceutically acceptable salt as a stand-alone therapy.

Alternatively, the subject with HR+/HER2− breast cancer may be treated with Compound (1) or its pharmaceutically acceptable salt in combination with a second breast cancer therapy. Here, Compound (1) or its pharmaceutically acceptable salt may be considered a “first breast cancer therapy” while a breast cancer therapy not based on administration of Compound (1) is referred to as a “second breast cancer therapy.” The second breast cancer therapy may involve administration of one or more anticancer agents (examples of which are described hereinafter) (other than Compound (1) or its salts) and/or non-drug therapies such as radiation therapy. Such combination therapy is intended to cover both simultaneously administered and sequentially (as pre- or post-treatment) administered combination therapy.

In terms of anticancer agent(s) administered as the second breast cancer therapy for the treatment of HR+/HER2− breast cancer, particular preference is given to endocrine therapy (ET) and/or cell cycle inhibitor therapy.

The endocrine therapy may include, but is not limited to, treatment using one or more of an aromatase inhibitor (e.g., anastrozole, letrozole, exemestane, vorozole, formestane, fadrozole), a selective estrogen receptor modulator (e.g., tamoxifen, 4-hydroxy tamoxifen, acolbifene, EM-800, toremifene, droloxifene, LY 117018, raloxifene, nafoxidine, and trioxifene), and a selective estrogen receptor degrader (e.g., fulvestrant, brilanestrant, elacestrant).

The cell cycle inhibitor therapy may include, but is not limited to, treatment with one or more cyclin-dependent kinase 4 and 6 (CDK4/6) inhibitors such as abemaciclib, palbociclib, and ribociclib.

Techniques for administering radiation therapy are known in the art, and these techniques can be used in the combination therapy described herein. The administration of Compound (1) in this combination therapy can be determined as described herein. Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachytherapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive microspheres.

The HR+ breast cancer may be a recurrent or refractory HR+ breast cancer, preferably a recurrent or refractory HR+/HER2− breast cancer.

Subjects with a recurrent or refractory cancer (e.g., breast cancer) who have previously undergone at least one treatment regimen with one or more anticancer agents, preferably at least two treatment regimens with at least two different anticancer agents may be treated with Compound (1) or its pharmaceutically acceptable salt. In some cases, the recurrent or refractory cancer may have acquired resistance to, or intractability from, the prior treatment regimen(s). For example, a subject with a HR+ cancer treated previously with one or more anticancer agents (e.g., endocrine therapy and/or a cell cycle inhibitor), and that have progressed on, failed to respond to, or relapsed from the prior treatment(s) with the anticancer agent(s), may develop resistance/intractability as a result of exposure of the cancer to the anticancer agent(s).

A preferred embodiment of the present disclosure involves administering Compound (1) or its pharmaceutically acceptable salt, either alone or in combination with one or more second breast cancer therapies, to a subject with recurrent or refractory HR+ breast cancer, preferably HR+/HER2− breast cancer, the subject having previously undergone at least one treatment with, and optionally acquired resistance to or intractability from, one or more of endocrine therapy (ET) and a cyclin-dependent kinase 4 and 6 (CDK4/6) inhibitor. For example, the cancer may be an endocrine therapy-resistant breast cancer such as an aromatase inhibitor-resistant cancer, a selective estrogen receptor modulator-resistant cancer, or a selective estrogen receptor degrader-resistant cancer, with specific mention being made to a tamoxifen-resistant breast cancer, a fulvestrant-resistant breast cancer, etc. In another example, the cancer may be a CDK4/6 inhibitor-resistant cancer such as a abemaciclib-resistant cancer, a palbociclib-resistant cancer, a ribociclib-resistant cancer, etc.

Resistance/intractability from the prior treatment(s) with ET may optionally manifest in the cancer in the form of ER downregulation; ligand-binding independent ER activation, for example, via estrogen receptor mutation (e.g., ESR1 mutations); downregulation of ER co-repressors such as via NF1 genetic variants; bypass signaling via the Ras pathway; dysregulated MAPK pathway and/or PI3K pathway signaling, for example from genetic aberrations in one or more components of these pathways such as upregulation of RSK, AKT, and/or S6K; or aberrations of any other cancer driver genes that result in loss-of-function of tumor suppressor genes/proteins or gain-of-function alterations in oncogenes/oncogene-encoded proteins. Resistance/intractability from the prior treatment(s) with one or more CDK4/6 inhibitors may optionally manifest in the cancer in the form of retinoblastoma (RB) gene/protein loss, a tumor suppressor and known driver of resistance to CDK4/6 inhibitors; PTEN loss of gene or variants which lead to loss of function; ESR1 mutation; NF1 genetic variant; dysregulated MAPK pathway and/or PI3K pathway signaling; or aberrations of any other cancer driver genes that result in loss-of-function of tumor suppressor genes/proteins or gain-of-function alterations in oncogenes/oncogene-encoded proteins. In particular, the recurrent or refractory HR+ breast cancer may harbor an NF1 genetic variant. When treating recurrent or refractory HR+ breast cancer with combination therapy, Compound (1) may be administered to revert resistance/sensitize the cancer to the co-administered anticancer agent(s).

Compound (1) or its pharmaceutically acceptable salt may also be used to treat estrogen deprived breast cancers, such as those which have acquired resistance through estrogen hypersensitivity or estrogen-independent activation of estrogen receptors.

Before commencing treatment, determination may be made as to whether the subject has progressed or relapsed from prior therapy (e.g., has recurrent or refractory cancer that has been previously treated with endocrine therapy (ET), a CDK4/6 inhibitor, etc.) and/or has one or more aberrations as identified above (e.g., NF1, PTEN, KRAS, etc.). Thus, the methods may involve a pre-screening step to determine whether the subject meets at least one of these criteria and is a good candidate for treatment. Such a determination may be made from analyzing family history of cancers involving the aberration(s), by genotyping the subject or analyzing any biological sample from the subject including blood or tumor samples taken from the subject using assays such as those described hereinafter, or from historical records or previous testing performed on the subject. If the subject is determined to have recurrent or refractory cancer, such as recurrent or refractory HR+/HER2− breast cancer, and/or harbors one or more genetic aberrations such as those described in the present disclosure, treatment with Compound (1) or its pharmaceutically acceptable salt is appropriate.

Predictive biomarkers which may be used to identify individuals who are likely to be responsive to treatment herein, include, but are not limited to, NF1 genetic variants, PTEN loss or mutations causing loss-of-function, and KRAS mutations (specifically KRAS G12X mutations). A companion diagnostic (CDx) test may be developed to analyze biological samples.

The subject's receptor status and genotype, including whether they harbor any gene/protein aberrations, may be determined, e.g., during subject pre-screening, via fresh biopsy, from previous testing performed on the subject, or otherwise confirmed according to known assays, including cleared or approved in vitro diagnostic (IVD) assays or assays for this purpose. Examples of which include, but are not limited to, testing by next generation sequencing (NGS)-based gene panel, whole exome profiling, polymerase chain reaction (PCR), in situ hybridization (ISH) including fluorescence in situ hybridization (FISH), immunohistochemistry (IHC), flow cytometry, or other assays that can determine receptor status or aberrations on tumor tissues or circulating tumor DNA (ctDNA), RNA, protein, etc. For example, subjects who do not have archival tumor tissue samples can be biopsied and the fresh tumor biopsy can be analyzed for confirmation of preexisting aberrations.

The terms “administer”, “administering”, “administration”, and the like, refer to the methods that may be used to enable delivery of the active ingredient to the desired site of biological action. Routes or modes of administration are as set forth herein. These methods include, but are not limited to, oral routes, intraduodenal routes, parenteral injection (including intravenous, subcutaneous, intraperitoneal, intramuscular, intravascular, or infusion), topical/transdermal, and rectal/vaginal administration. Those of ordinary skill in the art are familiar with administration techniques that can be employed. Oral administration is preferred.

In the present disclosure, the term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”.

Regarding the administration schedule of the present disclosure, “continuous” means administration every day without interruption during the treatment course. If the administration schedule follows an “intermittent” administration schedule, then days of administration may be followed by “rest days” or days of non-administration of drug within the course.

A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

The dosage amount and treatment duration are dependent on factors, such as bioavailability of a drug, administration mode, toxicity of a drug, gender, age, lifestyle, body weight, the use of other drugs and dietary supplements, the disease stage, tolerance and resistance of the body to the administered drug, etc., and then determined and adjusted accordingly. An appropriate dosage amount may differ from one individual to another. An appropriate dosage amount in any individual case may be determined using techniques, such as dose escalation.

The subject can be treated with Compound (1) or its pharmaceutically acceptable salt at, for example, dose levels for continuous (7 days of administration in a week) dosing of from about 1 mg/day, from about 5 mg/day, from about 10 mg/day, from about 15 mg/day, from about 20 mg/day, from about 30 mg/day, from about 40 mg/day, from about 50 mg/day, from about 60 mg/day, from about 77 mg/day, from about 80 mg/day, from about 100 mg/day, from about 120 mg/day, from about 160 mg/day, and up to about 1,500 mg/day, up to about 1,200 mg/day, up to about 960 mg/day, up to about 800 mg/day, up to about 640 mg/day, up to about 500 mg/day, up to about 400 mg/day, up to about 320 mg/day, up to about 300 mg/day, up to about 280 mg/day, up to about 250 mg/day, up to about 240 mg/day, up to about 200 mg/day, up to about 180 mg/day. The dosing level may be varied within the ranges such as from about 20 mg/day to about 960 mg/day, from about 40 mg/day to about 640 mg/day, and from about 80 mg to about 320 mg/day. The administration dose level can be changed during an administration schedule, for example, the administration can begin with low dose for some time and then increased, or the administration can begin with high dose for some time and then decreased.

The subject can be treated with Compound (1) or its pharmaceutically acceptable salt at, for example, dose levels for intermittent dosing of from about 20 mg/day, from about 40 mg/day, from about 60 mg/day, from about 80 mg/day, from about 100 mg/day, from about 200 mg/day, from about 300 mg/day, from about 400 mg/day, from about 500 mg/day, from about 600 mg/day, and up to about 3,000 mg/day, up to about 2,500 mg/day, up to about 2,000 mg/day, up to about 1,500 mg/day, up to about 1,000 mg/day, up to about 900 mg/day, up to about 800 mg/day, up to about 700 mg/day, or any dosing level within the ranges.

The dosing can be continuous (7 days of administration in a week) or intermittent, for example, depending the pharmacokinetics and a particular subject's clearance/accumulation of the drug. If intermittently, the schedule may be, for example, 4 days of administration and 3 days off (rest days) in a week or any other intermittent dosing schedule deemed appropriate using sound medical judgement. Continuous administration is preferred. The dosing can be performed once per day (QD) or more than once per day (b.i.d., t.i.d., etc.), with doses of about 20 to 960 mg/day QD being preferred. The daily dose may be administered as a single dose or multiple individual divided doses. For example, two (2) tablets, each tablet containing 40 mg of Compound (1) or its pharmaceutically acceptable salt, may be administered to the subject once per day (QD) for a total dose of 80 mg/day. In another example, two (2) tablets, each tablet containing 40 mg of Compound (1) or its pharmaceutically acceptable salt, may be administered to the subject twice per day (b.i.d.) for a total dose of 160 mg/day.

The dosing whether continuous or intermittent is continued for a particular treatment cycle, typically at least a 28-day cycle, which can be repeated with or without a drug holiday. Longer or shorter cycles can also be used such as 14 days, 18 days, 21 days, 24 days, 35 days, 42 days, 48 days, or any range therebetween. The cycle may be repeated without a drug holiday or with a drug holiday depending upon the subject. Other schedules are possible depending upon the presence or absence of adverse events, response of the cancer to the treatment, patient convenience, and the like. An “adverse event” refers to any unfavorable or unintended illness or symptom thereof occurring in a patient to whom a drug has been administered. It does not matter whether there is a causal relationship with the drug or not. For intermittent dosing, the dosing can be performed, for example, on day 1, 3, 5, 7, 9, 11, 13, 15, 17, 19, 21, 23, 25, and 27; on day 1, 4, 8, 11, 15, 19, 23, and 27; on day 1, 3, 5, 8, 10, 12, 15, 17, 19, 22, 24, and 26 in a 28 day cycle.

Such continuous or intermittent administration is applicable also to combination therapies where Compound (1) or its pharmaceutical acceptable salt is administered in combination with one or more other anticancer agents or non-drug therapies.

Compound (1) may be dosed using an up-titration regimen, whereby a subject is started with a low dose for a certain period of time (e.g., 2 weeks) and then the dose is escalated. The dose may be up-titrated until either a target or maximum dose is reached or the subject experiences adverse events at which point the escalation is stopped and the drug dosing is reduced to a previous dose where the adverse event was not experienced or was not serious enough to require stoppage of the treatment. A subject that experiences an adverse event may also be managed with dosing interruptions (e.g., a drug holiday), if deemed appropriate. Typical dosing for the continuous regimen is provided above, but higher or lower doses may be used depending on the subject's response to the treatment and presence or absence of adverse events. If a dose is well-tolerated, the dose can be increased. The administration may be continued for one cycle, e.g., 28 days, the cycle may then be repeated, as desired.

As described below, Compound (1) or its pharmaceutically acceptable salt may be specially formulated for administration in solid or liquid form, including those adapted for the following: (1) oral administration, for example, drenches (aqueous or non-aqueous solutions or suspensions), tablets or capsules, e.g., those targeted for buccal, sublingual, and systemic absorption, boluses, powders, granules, syrups, pastes for application to the tongue; (2) parenteral administration, for example, by subcutaneous, intramuscular, intravenous or epidural injection as, for example, a sterile solution or suspension, or sustained release formulation; (3) topical application/transdermal administration, for example, as a cream, ointment, or a controlled release patch or spray applied to the skin; (4) intravaginally or intrarectally, for example, as a pessary, cream or foam; or (5) nasally. In the case of Compound (1) or its pharmaceutically acceptable salt, an oral formulation is preferable.

Formulations can be prepared using a pharmaceutically acceptable carrier or the like by using known formulation methods. Pharmaceutically acceptable carriers are those materials, compositions, or vehicles, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier should be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, com oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations, such as cyclodextrins, liposomes, and micelle forming agents, e.g., bile acids, just to name a few.

Pharmaceutically acceptable carriers may be categorized as various general-purpose agents such as excipients, binders, disintegrating agents, lubricants, diluents, dissolution aids, suspending agents, swelling agents, isotonic agents, pH adjusters, buffers, stabilizers, colorants, flavoring agents, corrigents, and the like.

Examples of excipients include, but are not limited to, lactose, sucrose, D-mannitol, glucose, starch (corn starch), calcium carbonate, kaolin, microcrystalline cellulose, fumaric acid, and silicic acid anhydride.

Examples of binders include, but are not limited to, water, ethanol, 1-propanol, 2-propanol, simple syrup, liquid glucose, liquid a-starch, liquid gelatin, D-mannitol, carboxymethyl cellulose, hydroxypropyl cellulose (e.g., low viscosity hydroxypropyl cellulose), hydroxypropyl methylcellulose (hypromellose), hydroxypropyl starch, methyl cellulose, ethyl cellulose, shellac, calcium phosphate, polyvinylpyrrolidone.

Examples of disintegrants include, but are not limited to, low-substituted hydroxypropyl cellulose, dry starch, partially pregelatinized starch, crystalline cellulose, carmellose sodium, carmellose calcium, D-mannitol, crospovidone, croscarmellose sodium, sodium alginate, agar powder, sodium hydrogen carbonate, calcium carbonate, sodium lauryl sulfate, stearic acid monoglyceride, and lactose.

Examples of lubricants include, but are not limited to, hydrogenated oil, sucrose fatty acid ester, sodium lauryl sulfate, stearic acid, purified talc, sodium stearate, magnesium stearate, borax, and polyethylene glycol.

Examples of colorants include, but are not limited to, edible yellow No. 5 dye, edible blue No. 2 dye, edible lake dye, iron sesquioxide, yellow sesquioxide, and titanium dioxide.

Examples of sweetening/flavoring agents include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, oil of spearmint, oil of sassafras, oil of clove, cinnamon, anethole, menthol, thymol, eugenol, eucalyptol, lemon, lime, and lemon-lime.

If desired, an enteric coating or a coating to increase the persistence of effects can be provided by methods desirable for oral preparations. Examples of such coating agents include hydroxypropyl methylcellulose, ethyl cellulose, hydroxymethyl cellulose, hydroxypropyl cellulose, polyethylene glycol, and Tween 80 (registered trademark).

Compound (1) or its pharmaceutically acceptable salt are preferably formulated in solid dosage form for oral administration, such as in the form of capsules, tablets, pills, dragees, powders, granules, troches, and the like, with preference given to film-coated tablets. Compound (1) or its pharmaceutically acceptable salt may be mixed with one or more pharmaceutically acceptable carriers such as sodium citrate or dicalcium phosphate, and/or any of the following: (1) fillers or extenders, such as starches, lactose (e.g., lactose monohydrate), sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants (e.g., fatty acid esters of sorbitan and polyalkolyated fatty acid esters of sorbitan such as Tween 80 (registered trademark); (8) absorbents, such as kaolin and bentonite clay; (9) lubricants, such as talc, calcium stearate, magnesium stearate (e.g., vegetable derived), solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the formulations may also comprise pH adjusters or buffering agents. Solid compositions of a similar type may also be employed as fillers in soft and hard shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methylcellulose), lubricant, inert diluent, preservative, disintegrant (for example, sodium starch glycolate or cross-linked sodium carboxymethyl cellulose), surface active or dispersing agent. Molded tablets may be made by molding in a suitable machine a mixture of the powdered compound moistened with an inert liquid diluent. The tablets, and other solid dosage forms may optionally be scored or prepared with coatings and shells, such as enteric coatings and other coatings well known in the pharmaceutical formulating art. One example coating formulation may include hypromellose, polyethylene glycol, titanium dioxide, and optionally a coloring agent. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria retaining filter, or by incorporating sterilizing agents in the form of sterile solid compositions which can be dissolved in sterile water, or some other sterile injectable medium immediately before use. These formulations may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above described excipients.

Coated tablet dosage forms of Compound (1) or its pharmaceutically acceptable salt are preferred, such as those containing fumaric acid, lactose monohydrate, microcrystalline cellulose, croscarmellose sodium, magnesium stearate, hypromellose, polyethylene glycol, and titanium dioxide as inactive ingredients.

Compound (1) or its pharmaceutically acceptable salt may be formulated for parenteral administration, for intravenous, subcutaneous, intraperitoneal, intramuscular, intravascular, or infusion administration, by combining Compound (1) or its pharmaceutically acceptable salt with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and non-aqueous carriers which may be employed include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the desired particle size in the case of dispersions, and by the use of surfactants. These compositions may also contain adjuvants such as preservatives, wetting agents, emulsifying agents, dispersing agents, pH regulators, stabilizers, local anesthetics, etc. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The treatment methods of the present disclosure may involve administration of Compound (1) or pharmaceutically acceptable salt thereof as a stand-alone therapy. The treatment may also involve administration as a post-operative auxiliary chemotherapy that is performed to prevent recurrence of tumors after surgically removing tumors, as well as pre-operative auxiliary chemotherapy prior to surgery to surgically remove tumors. In some cases, such as with breast cancer, surgery may include a lumpectomy, a mastectomy, a breast reconstruction, and the like. In some cases, such as with lung cancer, surgery may include pneumonectomy, lobectomy, wedge resection, sleeve resection, thoracoscopy, and the like. The treatment may also include administration of Compound (1) or pharmaceutically acceptable salt thereof during or after radiation therapy or as an adjuvant therapy to prevent recurrence of the tumor in a patient where other treatments such as surgery have rendered the patient cancer-free.

Subjects may be treated herein whom have not previously undergone a treatment regimen with an anticancer agent(s), i.e., Compound (1) or its pharmaceutically acceptable salt are administered as first-line chemotherapy. Alternatively, as described heretofore, subjects may be treated whom have previously undergone a treatment regimen with one or more anticancer agents (other than Compound (1) or its salt forms) specific examples of which include, but are not limited to, an endocrine therapy and a CDK4/6 inhibitor. That is, Compound (1) or its pharmaceutically acceptable salt may be administered as second-, third-, fourth-, etc. line therapy.

Examples of anticancer agents include, but are not limited to, chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anticancer agents have been categorized within a specific group(s) or subgroup(s) herein, many of these agents can also be listed within one or more other group(s) or subgroup(s), as would be presently understood in the art. The anticancer agent is not particularly limited, and examples thereof include, but are not limited to, a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, an immune checkpoint inhibitor, an anti-PD-1 agent, an anti-PD-L1 agent, an anti-TIGIT agent, a colony-stimulating factor, an immunomodulator, an immunomodulatory imide (IMiD), an anti-CTLA4 agent, an anti-LAG1 agent, an anti-OX40 agent, a GITR agonist, a CAR-T cell, a BiTE, a signal transduction inhibitor, a growth factor inhibitor, a tyrosine kinase inhibitor, an EGFR inhibitor, a HER2 inhibitor, a histone deacetylase (HDAC) inhibitor, a proteasome inhibitor, a cell-cycle inhibitor, an anti-angiogenesis agent, a matrix-metalloproteinase (MMP) inhibitor, a hepatocyte growth factor inhibitor, a TOR inhibitor, a KDR inhibitor, a VEGF inhibitor, a HIF-1α inhibitor a HIF-2α inhibitor, a fibroblast growth factor (FGF) inhibitor, a RAF inhibitor, a MEK inhibitor, an ERK inhibitor, a PI3K inhibitor, an AKT inhibitor, an MCL-1 inhibitor, a BCL-2 inhibitor, an SHP2 inhibitor, a BRAF-inhibitor, a RAS inhibitor, a gene expression modulator, an autophagy inhibitor, an apoptosis inducer, an antiproliferative agent, and a glycolysis inhibitor.

Non-limiting examples of chemotherapeutic agents include mitotic inhibitors and plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trietylenephosphoramide, and trimethylolomelamine; ambamustine; bendamustine; dacarbazine; cyclophosphamide; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), tegafur/gimeracil/oteracil potassium, tegafur/uracil, trifluridine, trifluridine/tipiracil hydrochloride, 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine; 3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4(5)-imidazoles, EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

Non-limiting examples of immunotherapeutic agents (i.e., immunotherapy) include biological response modifiers, cytokine inhibitors, tumor vaccines, monoclonal antibodies, immune checkpoint inhibitors, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), ipilimumab, lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, veltuzumab, and trastuzumab.

Non-limiting examples of immune checkpoint inhibitors include anti-PD-1 agents or antibodies such as cemiplimab, zimberelimab, nivolumab, and pembrolizumab; anti-PD-L1 agents or antibodies such as atezolizumab, avelumab, and durvalumab; anti-TIGIT agents or antibodies such as tiragolumab and domvanalimab; anti-CTLA-4 agents or antibodies such as ipilumumab and tremelimumab; anti-LAG1 agents; and anti-OX40 agents.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, HER2 inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, RAF inhibitor, MEK inhibitors, ERK inhibitors, PI3K inhibitors, AKT inhibitors, MCL-1 inhibitors, BCL-2 inhibitors, SHP2 inhibitors, BRAF-inhibitors, RAS inhibitor, heat shock protein (HSP) 90 inhibitor, gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors (i.e., other than Compound (1) or its salt), anlotinib, avapritinib, axitinib, dasatinib, dovitinib, imatinib, lenvatinib, lonidamine, nilotinib, nintedanib, pazopanib, pegvisomant, ponatinib, vandetanib, and EGFR and/or HER2 inhibitory agents.

Non-limiting examples of EGFR inhibitors include small molecule antagonists of EGFR such as afatinib, brigatinib, erlotinib, gefitinib, lapatinib, neratinib, dacomitinib, vandetanib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1:1311-1318; Huang, S. M., et al., 1999, Cancer Res. 15:59(8):1935-40; and Yang, X., et al., 1999, Cancer Res. 59:1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of HER2 inhibitors include HER2 tyrosine kinase inhibitors such as afatinib, lapatinib, neratinib, and tucatinib; and anti-HER2 antibodies or drug conjugates thereof such as trastuzumab, trastuzumab emtansine (T-DM1), pertuzumab, margetuximab, trastuzumab deruxtecan (DS-8201a), and trastuzumab duocarmazine. Non-limiting examples of FGFR inhibitors (FGFR-TKI) include anlotinib, ponatinib, dovitinib, lucitanib, lenvatinib, nintedanib, erdafitinib (JNJ-42756493), infigratinib (BGJ398), pemigatinib (INCB054828), rogaratinib (BAY1163877), derazantinib (ARQ 087), futibatinib (TAS-120), LY2874455, AZD4547, Debio 1347, and fisogatinib (BLU-554).

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

Non-limiting examples of anti-angiogenic agents (or angiogenesis inhibitors) include, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478; HIF-2α inhibitors such as belzutifan and the HIF-2α inhibitors described in WO 2015/035223; fibroblast growth factor (FGF) or FGFR inhibitory agents such as B-FGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. No. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. No. 5,863,949, U.S. Pat. No. 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borean, Denmark), and VEGF-TRAP™.

Other anti-angiogenic agents may include, but are not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-111142, GCS-100, GW 2286 (GlaxoSmithKline, UK), IL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and MedImmune, US), MAb VEGF (Xenova, UK), marimastat, maspin (Sosei, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exelixis, US), ziv-aflibercept, and ZD 6126.

The anticancer agent(s) that may be combined with Compound (1) may also be an active agent that disrupts or inhibits RAS-RAF-ERK or PI3K-AKT-TOR signaling pathways or is a PD-1 and/or PD-L1 antagonist. Examples of which include, but are not limited to, a RAF inhibitor, an EGFR inhibitor, a MEK inhibitor, an ERK inhibitor, a PI3K inhibitor, a AKT inhibitor, a TOR inhibitor, an MCL-1 inhibitor, a BCL-2 inhibitor, a SHP2 inhibitor, a proteasome inhibitor, or an immune therapy, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and ASTX029.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE-477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; taselisib (GDC-0032); and ZSTK474.

Non-limiting examples of AKT inhibitors include Akt-1-1 (inhibits Aktl) (Barnett et al., (2005) Biochem. J., 385 (Pt. 2), 399-408); Akt-1-1,2 (Barnett et al., (2005) Biochem. J. 385 (Pt. 2), 399-408); API-59CJ-Ome (e.g., Jin et al., (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 3493S-3498S); perifosine, Dasmahapatra et al., (2004) Clin. Cancer Res. 10(15), 5242-52, 2004); phosphatidylinositol ether lipid analogues (e.g., Gills and Dennis (2004) Expert. Opin. Investig. Drugs 13, 787-97); triciribine (Yang et al., (2004) Cancer Res. 64, 4394-9); imidazooxazone compounds including trans-3-amino-1-methyl-3-[4-(3-phenyl-5H-imidazo[1,2-c]pyrido[3,4-e][1,3] oxazin-2-yl)phenyl]-cyclobutanol hydrochloride (WO 2012/137870); afuresertib;; capivasertib; 8-[4-(1-aminocyclobutyl)phenyl]-9-phenyl-1,2,4-triazolo[3,4-f][1,6]naphthyridin-3(2H)-one (MK2206) and pharmaceutically acceptable salts thereof; AZD5363; trans-3-amino-1-methyl-3-(4-(3-phenyl-5H-imidazo[1,2-c]pyrido[3,4-e][1,3]oxazin-2-yl)phenyl)cyclobutanol (TAS-117) and pharmaceutically acceptable salts thereof; and patasertib.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-[3-hydroxy(hydroxymethyl)methylpropanoate]-rapamycin; 40-epi-(tetrazolyl)-rapamycin (also called ABT578); AZD8055; 32-deoxorapamycin; 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. No. 5,118,677, U.S. Pat. No. 5,118,678, U.S. Pat. No. 5,100,883, U.S. Pat. No. 5,151,413, U.S. Pat. No. 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and S63845.

Non-limiting examples of SHP2 inhibitors include JAB-3068, RMC-4630, TNO155, SHP-099, RMC-4550, and SHP2 inhibitors described in WO 2019/167000, WO 2020/022323 and WO2021/033153.

Non-limiting examples of RAS inhibitors include AMG510 (sotorasib), MRTX849 (adagrasib), LY3499446, JNJ-74699157 (ARS-3248), ARS-1620, ARS-853, GDC-6036, D-1553, JDQ433, JAB-21822, RM-007, RM-008, MRTX1133, and KRpep-2d. Non-limiting examples of HSP90 inhibitors include pimitespib.

Additional non-limiting examples of anticancer agents that may be suitable for use include, but are not limited to, 2-ethylhydrazide, 2,2′,2″-trichlorotriethylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflornithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole+fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone+pentazocine, nitracrine, nolatrexed, NSC 631570 octreotide (Ukrain), olaparib, P-30 protein, PAC-1, palifermin, pamidronate, pamidronic acid, pentosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

As used in the present disclosure, the term “combination,” “combined,” or a variation thereof is intended to define a therapy involving the use of two or more compound/drug combinations. The term can refer to compounds/drugs that are administered as part of the same overall dosage schedule. The respective dosages of two or more compounds/drugs can be different. The combination therapy is intended to embrace administration of these compounds/drugs in a sequential manner, that is, wherein each compound/drug is administered at a different time (e.g., a first drug is administered as a pre-treatment or a post-treatment with reference to a second drug of the combination), as well as administration of these compounds/drugs, or at least two of the compounds/drugs, in a substantially simultaneous manner. Substantially simultaneous administration can be accomplished, for example, by administering to the subject a single dosage form having a fixed ratio of each compound/drug or in multiple, single dosage forms for each of the compounds/drugs. Sequential or substantially simultaneous administration of each compound/drug can be effected by any appropriate route including, but not limited to, oral routes, intravenous routes, intramuscular routes, and direct absorption through mucous membrane tissues (e.g., buccal). The compounds/drugs can be administered by the same route or by different routes. For example, a first compound/drug of the combination selected may be administered by intravenous injection while the other compound/drug of the combination may be administered orally. Alternatively, for example, all compounds/drugs may be administered orally or all compounds/drugs may be administered by intravenous injection.

Combination therapy also can embrace the administration of the compounds/drugs as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of compound/drug and non-drug treatment is achieved. For example, in appropriate cases, the beneficial effect is still achieved when the non-drug treatment is temporally removed from the administration of the compound/drug, perhaps by days or even weeks.

### EXAMPLES

### Example 1: Evaluation of the Effect of NF1 Depletion on MAPK and P13K Pathways in ER+/HER2− Breast Cancer Cells

To investigate the effect of NF1 depletion on cell growth signals in ER+/HER2− breast cancer cells, the NF1 siRNA was transfected into the human ER+/HER2− breast cancer derived MCF7 cells, and the changes in the phosphorylation status of molecules which are representative markers of MAPK and PI3K signaling activities were confirmed by immunoblotting.

MCF7 cells cultured in phenol red-free RPMI1640 medium containing 10% charcoal stripped FBS were transfected with NF1 siRNA to a final concentration of 10 μmol/L using Lipofectamine RNAiMAX. The cells were then incubated for 1 day at 37° C., 5% CO₂. After that, E2 (estrogen) was added to the medium at 1 nmol/L final concentration, and the cells were cultured for 3 days, after which the cells were collected and cell extracts were prepared.

The expression of the following protein molecules were compared between NF1 knockdown cells and control cells by immunoblotting. (1) NF1 for confirmation of the knockdown of NF1, (2) pAKT Ser473, pERK Thr202/Tyr204, and pS6RP Ser235/236 for monitoring changes the activity of MAPK and PI3K signaling, (3) ERα for monitoring responsiveness of cells to estrogen-stimuli.

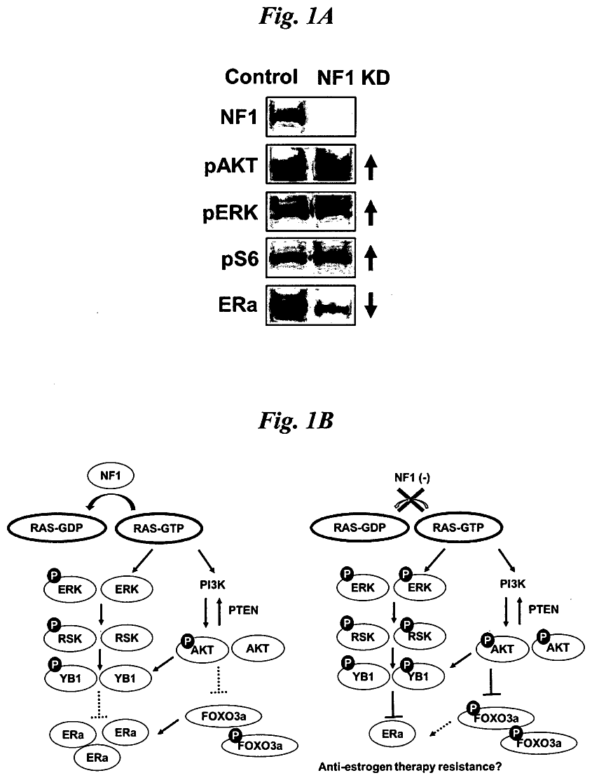

As shown in FIG. 1A, compared with control MCF7 cells, the phosphorylation levels of pAKT, pERK, and pS6 were increased in NF1 knockdown cells. These results indicate that simultaneous enhancement of MAPK and PI3K signaling due to NF1 downregulation or dysfunction by NF1 gene alterations. In addition, ERα expression was also decreased, suggesting that sensitivity to ER-targeting drugs may be reduced. These findings suggest that ER-targeted drugs are less effective in NF1-deficient ER+/HER2− breast cancer cells and that inhibition of MAPK and PI3K signaling pathways is more effective.

### Example 2: Evaluation of Growth Inhibition Effect of Compound (1) in the NF1 Depleted ER+/HER2− Breast Cancer Cells

To investigate the growth inhibition effect of Compound (1) on the NF1-depleted ER+/HER2− breast cancer cells, MCF7 and T47D, the serially diluted Compound (1) was exposed to the cells for 3 days as in the previous experiment.

The growth inhibition curve in MCF7 and T47D cells were obtained and the half maximal inhibitory concentration (IC50) value of growth inhibition were calculated as shown in Table 1.

TABLE 1

| | | Genetic alteration | | Compound (1) | |
|---|---|---|---|---|---|
| | | | PIK3CA/ | IC₅₀ (nmol/L) | |
| Name of cell line | Description | NF1 | PTEN | E2 (−) | E2 (+) |
| MCF7 | ER+, breast | WT | p.E545K/WT | 89 | 108 |
| MCF7_NF1KD | ER+, breast | KD | | 294 | 188 |
| T47D | ER+, breast | WT | p.H1047R/WT | 125 | 65 |
| T47D_NF1KD | ER+, breast | KD | | 111 | 37 |

Abbreviations: WT: Wild Type; KD: Knocked Down

As a result, Compound (1) showed a relatively strong IC50 value even in the presence or absence of estrogen in NF1 knockdown MCF7 and T47D cells. These results suggest that Compound (1) may have a significant growth inhibition effect against breast tumors with NF1 abnormalities.

### Example 3: Evaluation of Growth Inhibition Effect of Compound (1) in the NF1 Mutant Cell Lines

NF1 gene alteration has been reported not only in ER+/HER2− breast cancer but also in other breast cancer subtypes, such as HER2+ and triple negative breast cancer. It has also been found in colorectal cancer, melanoma, and lung cancer, and NF1 abnormality may be a cause of deregulated growth potential in some cancers.

In order to investigate the growth inhibitory effect of Compound (1) in NF1 mutant cancer cell lines, the IC50 values of growth inhibition assay were obtained and compared then in Table 2.

TABLE 2

| | | Genetic alteration | | | IC₅₀ (nmol/L) | | |
|---|---|---|---|---|---|---|---|
| | | | PIK3CA/ | | Compound | | |
| Name of cell line | Description | NF1 | PTEN | KRAS/BRAF | (1) | Alpelisib | Trametinib |
| BT483 | ER+, breast | p.R1204W | p.E542K/WT | WT/WT | 128 | 179 | >2000 |
| MDA-MB-175VII | ER+, breast | p.1679Dfs*21 | WT/WT | WT/WT | 70 | 1092 | 40 |
| HCC202 | HER2+, breast | p.X465_splice | p.E545K/WT | WT/WT | 157 | 918 | >2000 |
| MDA-MB-231 | TN, breast | p.T467Hfs*3 | WT/WT | p.G13D/ | 2807 | >10000 | >10000 |
| | | | | p.G464V | | | |
| HCT116 | Colon | p.P678Rfs*10, | p.H1047R/WT | p.G13D/WT | 355 | 6341 | 16 |
| | | p.P388T | | | | | |
| RKO | Colon | p.Y628Tfs*3, | p.H1047R/WT | WT/p.V600E | 118 | 2695 | 40 |
| | | p.V2205A, | | | | | |
| | | p.N2341Tfs*5 | | | | | |
| H1838 | Lung | p.N184Qfs*17 | WT/WT | WT/WT | 344 | 6414 | >2000 |
| MeWo | Melanoma | p.Q1336* | WT/WT | WT/WT | 844 | >10000 | 176 |

Abbreviations:
WT: Wild Type;
TN: Triple Negative

In this experiment, to compare the growth inhibitory effect of Compound (1) with those of MAPK and PI3K signaling inhibitors, the IC50 values of trametinib (as a MEK inhibitor), and Alpelisib (as a PI3K inhibitor) were obtained and compared.

Several NF1 mutant cell lines were found to be insensitive to MEK and/or PI3K inhibitors, with IC50 values ranging from 16 to more than 10,000 nmol/L for trametinib, and 179 to more than 10,000 nmol/L for Alpelisib. On the other hand, the IC50 value of Compound (1) was less than 1 μmol/L except for MDA-MB-231 cells, indicating that Compound (1) is more potent against NF1 mutant cell growth than drugs inhibiting either signaling pathway alone.

### Example 4: Evaluation of Target Inhibition and Apoptosis Induction by Compound (1) in NF1 Depleted ER+/HER2+ Breast Cancer Cell Lines

To investigate the mechanism of action of Compound (1) in inhibiting the growth of NF1 knockdown breast cancer cells, Compound (1), trametinib, Alpelisib, and fulvestrant were exposed to NF1-knockdown MCF7 and T47D cells at indicated final concentration for 24 hours. NF1, pPRAS40 Thr246, pYB1 Ser102, pS6RP Ser235/236, ERα, and cleaved PARP were detected by immunoblotting.

PRAS40, YB1, and S6RP are specific phosphorylation substrates of AKT, p90RSK, and p70S6K kinases, respectively, and the reduction of these phosphorylation signals indicates that target inhibition by Compound (1) occurs.

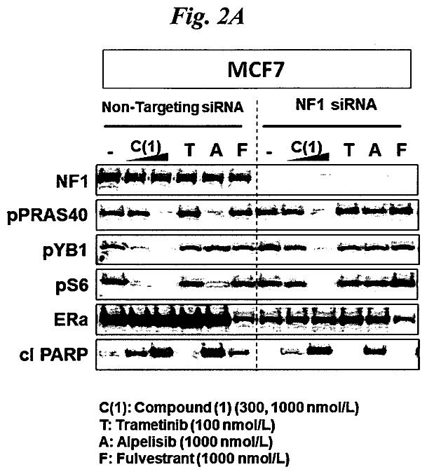

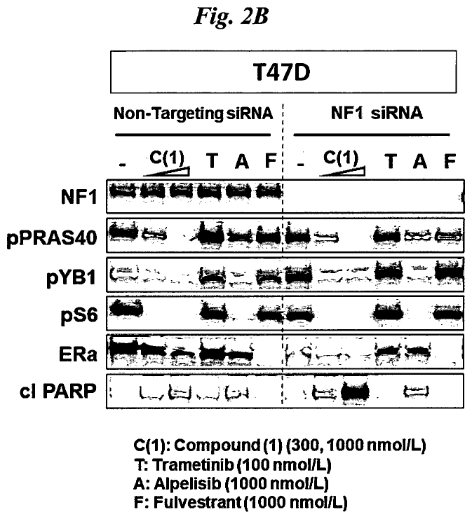

As shown in FIGS. 2A-2B, Compound (1) decreased the signals of pPRAS40, pYB1, and pS6RP in a concentration-dependent manner in both cells. These target inhibitions were also observed in the NF1 knockdown cells, indicating that Compound (1) is effective even in the cells with activated MAPK and PI3K signaling by NF1 abnormality as described in FIG. 1A.

The cleaved PARP signal serves as a marker to detect the apoptosis induction. In MCF7 cells, Alpelisib induced apoptosis in control cells (non-targeting siRNA) even as a single agent, but the induction of apoptosis is found to be attenuated in NF1 knockdown cells. Compound (1) single agent treatment also induced apoptosis in control cells (non-targeting siRNA cells) of MCF7, but in contrast to Alpelisib, Compound (1) did not exhibit the attenuation of apoptosis induction in NF1 knockdown cells. Further, in T47D cells, the effect of induction of apoptosis by Compound (I) was observed in a remarkably higher level in NF1 knockdown cells than control cells (non-targeting siRNA cells). Such significantly higher level of the apoptosis-inducing effect in NF1 knockdown cells compared to control cells were not observed in Alpelisib-treated group. These findings indicate that Compound (1) has a promising pharmacological effect on the antitumor activity against NF1-deficient breast cancer cells.

### Example 5: Evaluation of Antitumor Effect of Compound (1) in Combination with Fulvestrant in the Presence and Absence of Estrogen and NF1

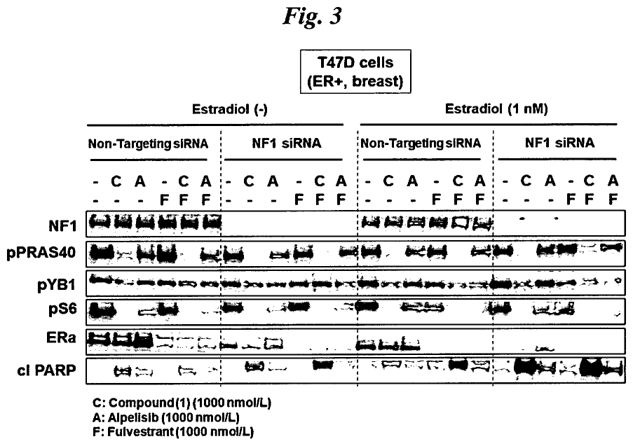

The actual standard of care for ER+/HER2− breast cancer uses the adjuvant therapies such as tamoxifen, letrozole, or fulvestrant. Patients with recurrent or refractory tumors are treated with additional use of CDK4/6 inhibitors or Alpelisib for PIK3CA-mutated cancers. As shown in FIG. 3, it was evaluated whether the antitumor effect of Compound (1) could be enhanced by the combination with fulvestrant in the combination of two types of conditions which promotes cancerous proliferation, (1) presence and absence of estrogen treatment, and (2) NF1 as a wildtype status and siRNA-targeted NF1 knockdown status.

Apoptosis induction by Compound (1) was observed in the presence or absence of estrogen treatment, both in NF1 wildtype cells and NF1 knockdown cells. By the treatment with fulvestrant alone, ER reduction was observed clearly, but had little apoptosis inducing activity in all fulvestrant treated groups.

However, in combination with Compound (1) and fulvestrant, apoptosis induction was enhanced remarkably in the presence of estrogen treatment in NF1 knockdown cells compared to fulvestrant mono-treatment cells. From these results, Compound (1) is potentially available to use in combination with fulvestrant for growth inhibition of ER+/HER2− breast cancer cells.

### Example 6: Exploration of Genetic Factors Correlated with Susceptibility Using Cell Panels

Cell growth inhibition assay was conducted according to 72 hour-standard protocol by CellTiter Glo 2.0 Assay.

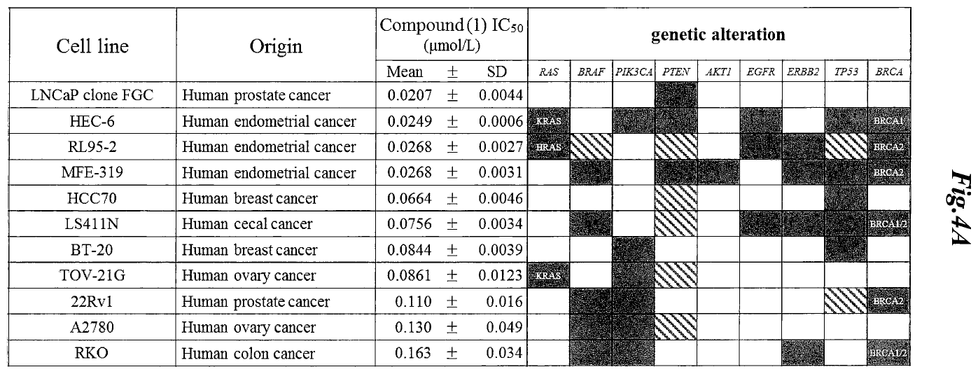

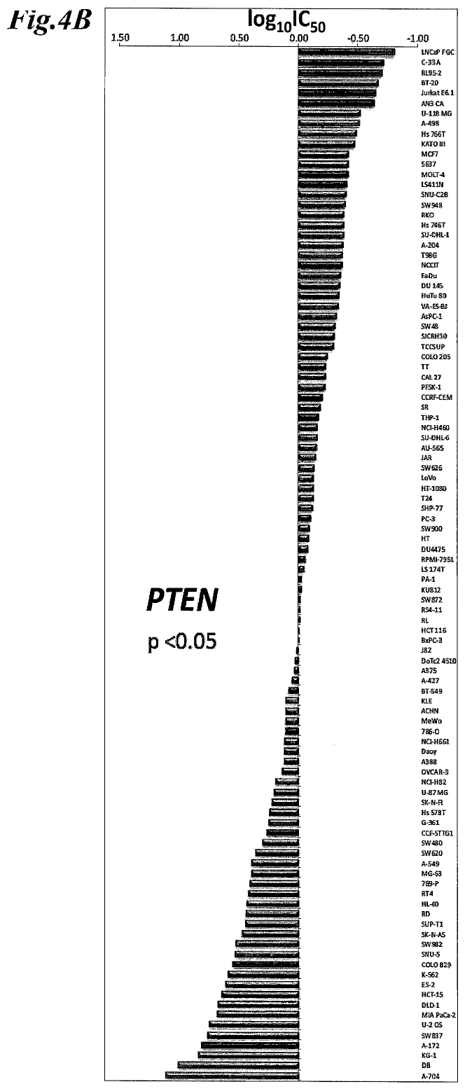

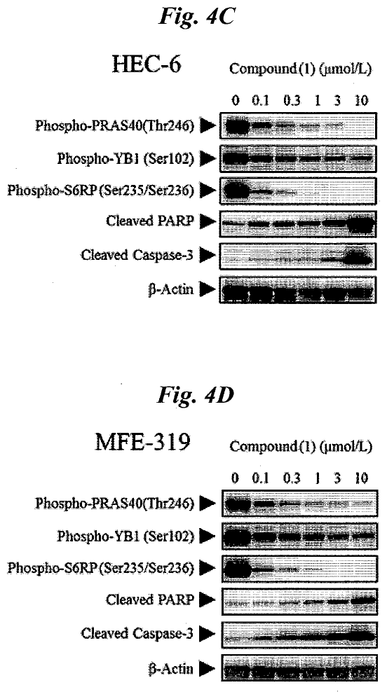

Compound (1) showed potent growth inhibition in cancer cell lines derived from various human cancer origins (FIG. 4A). Low IC50 value of Compound (1) indicates remarkable potency to inhibit growth of the resistant cells harboring oncogenic gene alterations, e.g. driver gene mutation (KRAS, EGFR, ERBB2, and PIK3CA) and/or deficiency of tumor suppressor gene (PTEN and TP53). Among these genetic alterations, cells with genetic variant of PTEN gene appeared to correlate with the strong IC50 value of Compound (1). To confirm the correlation between IC50 of Compound (1) and PTEN gene alteration, the large scale cell panel analysis has been conducted. In FIG. 4B, the correlation between the sensitivity to Compound (1) and PTEN genetic alterations was clearly observed. For further confirmation of the sensitivity of the cells with PTEN alteration to Compound (1), dose-dependent apoptosis induction was evaluated by monitoring the levels of cleaved caspase-3 and cleaved PARP using PTEN-mutated HEC-6 and MFE-319 cells treated for 48 hour after addition of serially diluted Compound (1) (FIGS. 4C-4D). Treatment of both cell lines with Compound (1) increased cleaved caspase-3 and cleaved PARP in a concentration dependent manner compared to control. These results suggest that Compound (1) induces apoptosis in the PTEN-mutated human cancer cells through the inhibition of target kinases.

### Example 7: Evaluation of the Antitumor Effect of Compound (1) or Sotorasib or the Combination of Compound (1) and Sotorasib in Nude Mice Bearing the KRAS_G12C and PIK3CA_K111E Mutated SW1573_Human Lung Tumor Xenografts

The SW1573 cell line from lung cancer was reported to have KRAS G12C mutation but low sensitivity to sotorasib (also called as AMG510), a G12C inhibitor (see also later Example 11).

In order to evaluate the antitumor effect of Compound (1) against the SW1573 tumors subcutaneously transplanted in nude mice, a comparison of the efficacy of Compound (1) with AMG510 alone and the combined efficacy of AMG510 plus Compound (1) have been conducted.

Dosing fluid containing Compound (1) using 0.5% w/v hydroxypropyl methylcellulose (HPMC) supplemented hydrochloric acid to achieve final concentration of 0.1 N were prepared. 30 mg/kg and 60 mg/kg of Compound (1) for single agent treatment were prepared and administered PO QD for 14 days. The antitumor effect of AMG510 was also evaluated at 30 mg/kg using 0.5% w/v HPMC by PO QD for 14 days. The combination treatment with 60 mg/kg Compound (1) and 30 mg/kg AMG510 by oral gavage every day for 14 days.

At the evaluation on Day 15, all groups treated with Compound (1) exhibited significant tumor growth inhibition compared with the control group (P<0.01 for 30 mg/kg, and P<0.001 for 60 mg/kg, Dunnett's test). The 30 mg/kg AMG510-treated group also exhibited significant tumor growth inhibition at almost the same level as 30 mg/kg of Compound (1). The combination group with Compound (1) and AMG510 exhibited significant tumor growth inhibition (P<0.001, Dunnett's test).

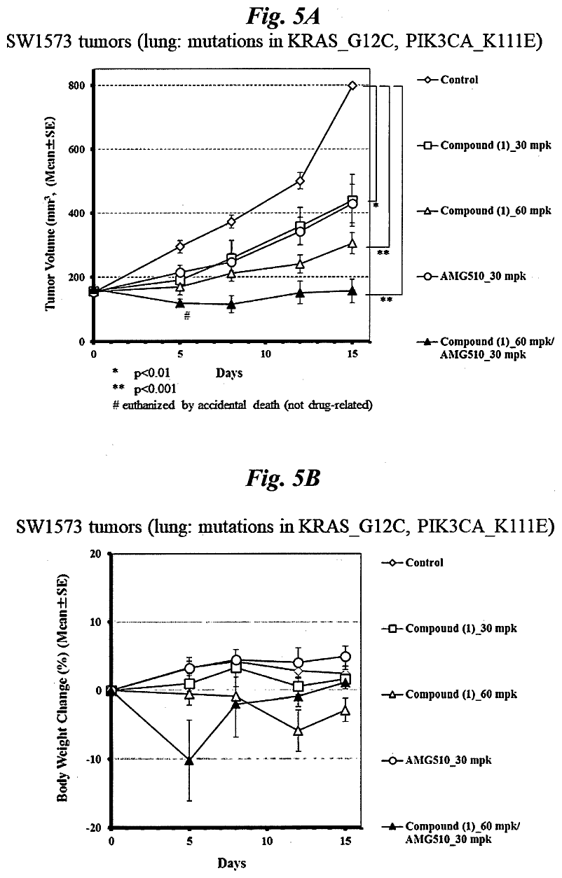

The results are summarized in FIGS. 5A-5B and Table 3.

TABLE 3
Antitumor effect of Compound (1) and AMG510 in mice with SW1573 xenograft tumors

| | | | | Number | Number | Tumor volume (mm³) | | p-value | T/C |
|---|---|---|---|---|---|---|---|---|---|
| | Dose | Dosing | | of | of | Day 0 | Day 15 | vs | ratio |
| Group | (mg/kg/day) | schedule | Route | animals | deaths | Mean ± SE | Mean ± SE | control | (%) |
| Control | 0 | qd, Day 1-14 | po | 5 | 0 | 152.3 ± 7.1 | 798.8 ± 7.4 | — | 100 |
| Compound (1) | 30 | qd, Day 1-14 | po | 5 | 0 | 157.2 ± 8.9 | 438.6 ± 81.1 | p < 0.01 | 55 |
| Compound (1) | 60 | qd, Day 1-14 | po | 5 | 0 | 155.4 ± 8.8 | 305.6 ± 33.0 | p < 0.001 | 38 |
| AMGS10 | 30 | qd, Day 1-14 | po | 5 | 0 | 153.5 ± 7.5 | 429.2 ± 60.2 | p < 0.01 | 54 |
| Compound (1)/AMG510 | 60/30 | qd, Day 1-14/qd, | po/po | 4* | 1 | 164.2 ± 11.4 | 156.4 ± 37.0 | p < 0.001 | 20 |
| | | Day 1-14 | | | | | | | |

Abbreviations: qd, quaque die (once daily);
po, per os (orally);
SE, standard error;
T/C ratio, treatment/control ratio of the mean tumor volume;
NA, not applicable
Note:
Tumor volume (mm3) = (major axis [mm]) × (minor axis [mm]) × (minor axis [mm])/2
T/C ratio (%) = (mean tumor volume of each treated group on Day 15)/(mean tumor volume of the control group on Day 15) × 100
Compound (1) and AMG510 and Trametinib was administered orally once daily for 14 days.
Statistical analysis was performed with Dunnett's test to compare the Day 15 tumor volume of each treated group with that of the control group.
*accidental deth n = 1

The T/C ratios of the groups treated with 30, 60 mg/kg of Compound (1), 30 mg/kg of AMG510, and combination of Compound (1) and AMG510 were 55%, 38%, 54%, and 20%, respectively. No mice had >20% loss in body weight from Day 0 over the experiment except for a case of accidental death due to dosing error in combination arm. These results demonstrated that Compound (1) showed efficacy as same or superior level than that of AMG510 in KRAS G12C mutant cells which is also carrying PIK3CA mutation. Additionally, the combination of Compound (1) and AMG510 also demonstrated clear enhancement of the antitumor efficacy by each agent alone with acceptable tolerability.

### Example 8: Evaluation of the Antitumor Effect of Compound (1) or Sotorasib or the Combination of Compound (1) and Sotorasib in Nude Mice Bearing the KRAS_G12C Mutated LU65_Human Lung Tumor Xenografts

To investigate the antitumor efficacy of Compound (1) against KRAS G12C mutated LU65 human lung tumors, 80 mg/kg/day of Compound (1), 30 mg/kg/day of AMG510, and their combination have been administered to male BALB/c nude mice subcutaneously implanted with LU65 tumor xenograft. Both Compound (1) and AMG510 alone and the combination of them were dosed every day by oral gavage. The tumor volumes and body weights of animals were recorded.

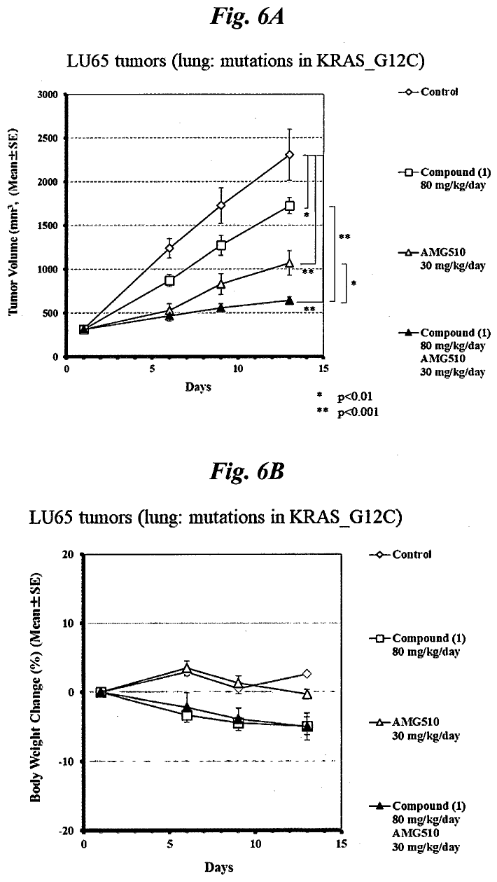

The results are summarized in FIGS. 6A-6B and Table 4 (evaluated at Day 13).

TABLE 4
Antitumor effect of Compound (1) and AMG510 in mice with LU65 xenograft tumors

| | | | | | | | | p-value | | | |
|---|---|---|---|---|---|---|---|---|---|---|---|
| | | | | Number | Number | Tumor volume (mm³) | | | vs | | T/C |
| | Dose | Dosing | | of | of | Day 1 | Day 13 | vs | Com- | vs | ratio |
| Group | (mg/kg/day) | schedule | Route | animals | deaths | Mean ± SE | Mean ± SE | Control | pound (1) | AMG510 | (%) |
| Control | — | — | — | 5 | 0 | 319.9 ± 26.0 | 2305.7 ± 293.7 | — | — | — | 100 |
| Compound (1) | 80 | qd, Day 1-21 | po | 5 | 0 | 311.6 ± 30.5 | 1724.3 ± 91.1 | <0.05 | — | — | 75 |
| AMG510 | 30 | qd, Day 1-21 | po | 5 | 0 | 313.4 ± 29.4 | 1067.9 ± 139.4 | <0.001 | — | — | 46 |
| Compound (1) + | 80 + 30 | qd, Day 1-21 + | po + po | 5 | 0 | 310.8 ± 34.2 | 643.4 ± 37.7 | <0.001 | <0.001 | <0.05 | 28 |
| AMG510 | | qd, Day 1-21 | | | | | | | | | |

Abbreviations: qd, quaque die (once daily);
po. per os (orally);
SE, standard error;
T/C ratio, treatment/control ratio of the mean tumor volume;
NA, not applicable
Note:
Tumor volume (mm³) = (major axis [mm]) × (minor axis [mm]) × (minor axis [mm])/2
T/C ratio (%) = (mean tumor volume of each treated group on Day 13)/(mean tumor volume of the control group on Day 13) × 100
Compound (1) and AMG510 and Trametinib afatinib were administered orally once daily for 12 days.
Statistical analysis was performed with Dammnett's rest to compare the Day 13 tumor volume of each treated group with that of the control group.

AMG510, a G12C inhibitor, significantly inhibited tumor growth (T/C: 46%, p<0.001), whereas Compound (1) showed a limited antitumor effect (T/C: 75%, p<0.01) even at a dose of 80 mg/kg. A significant combination effect (T/C: 28%, p<0.05 versus AMG510 alone, p<0.001 versus Compound (1) treatment alone) was observed. In addition, body weight changes were limited throughout the study period, indicating that Compound (1) was well tolerated as a single agent or in combination with AMG510.

### Example 9: Evaluation of the Antitumor Effect of Compound (1) in Nude Mice Bearing the KRAS_G12D, PIK3CA_H1047R, and PTEN_167K Mutated LS180 Human Colorectal Tumor Xenografts

The oncogenic KRAS mutations are diverse, including not only G12C but also G12X and G13X in the mutation hotspot Glycine at 12. To determine whether Compound (1), which targets downstream of MAPK and PI3K signaling, is effective against G12D mutated cancers, an efficacy experiment of Compound (1) was conducted using subcutaneously transplanted LS180 tumor derived from colorectal cancers with KRAS G12D and PIK3CA and PTEN gene alterations.

Evaluation was conducted by comparing the efficacy of 80 mg/kg/day of Compound (1) with that of 1 mg/kg/day MEK inhibitor trametinib used as a reference group.

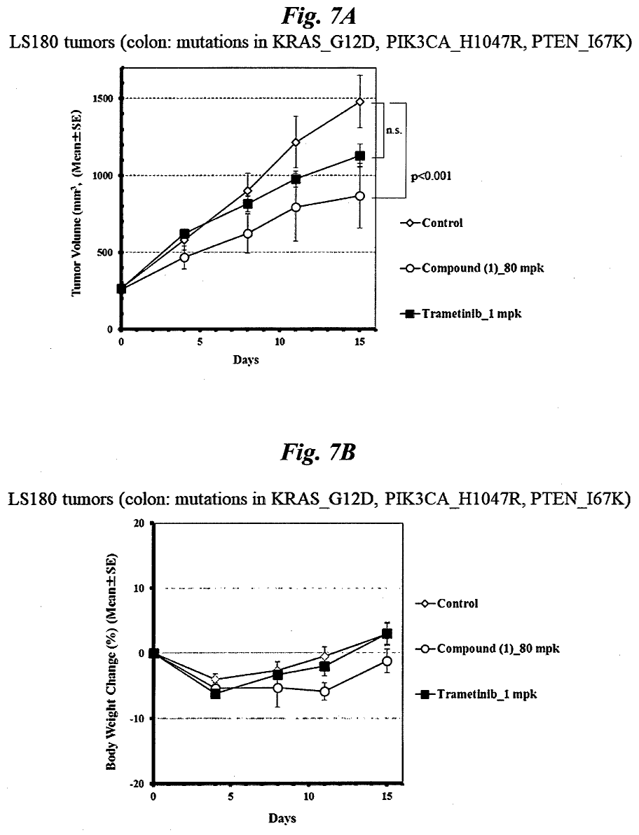

The results are presented in FIGS. 7A-7B and Table 5 (evaluated at Day 15).

TABLE 5
Antitumor effect of Compound (1) and Trametinib in mice with LS180 xenograft tumors

| | | | | Number | Number | Tumor volume (mm³) | | p-value | T/C |
|---|---|---|---|---|---|---|---|---|---|
| | Dose | Dosing | | of | of | Day 0 | Day 15 | vs | ratio |
| Group | (mg/kg/day) | schedule | Route | animals | deaths | Mean SE | Mean ± SE | control | (%) |
| Control | 0 | qd, Day 1-14 | po | 5 | 0 | 267.9 ± 26.5 | 1480.0 ± 169.7 | — | 100 |
| Compound (1) | 80 | qd, Day 1-14 | po | 5 | 0 | 258.5 ± 20.7 | 867.8 ± 210.6 | p < 0.01 | 59 |
| Trametinib | 1 | qd, Day 1-14 | po | 5 | 0 | 263.8 ± 20.2 | 1129.3 ± 73.5 | n.s. | 76 |

Abbreviations: qd, quaque die (once daily);
po, per os (orally);
SE, standard error;
T/C ratio, treatment/control ratio of the mean tumor volume;
NA, not applicable
Note:
Tumor volume (mm3) = (major axpound (1)_80 mpk
T/C ratio (%) = (mean tumor volume of each treated group on Day 15)/(mean tumor volume of the control group on Day 15) × 100
Compound (1) and Trametinib was administered orally once daily for 14 days.
Statistical analysis was performed with Dunnett's test to compare the Day 15 tumor volume of each treated group with that of the control group.

Compound (1) showed moderate but significant antitumor efficacy (T/C: 59%, p<0.01) and tolerability, while trametinib, which inhibits MAPK signaling activated by KRAS mutation, did not show sufficient efficacy (T/C: 76%, not significant by Dunnett's test).

These results suggest that MAPK inhibition alone may have limited efficacy in KRAS G12D mutated cancers with PIK3CA and/or PTEN genetic alterations. On the other hand, Compound 1 showed superior efficacy and tolerability compared to MAPK signal inhibition alone.

### Example 10: Evaluation of Antitumor Effect of Compound (1) or Trametinib or the Combination of Compound (1) and Trametinib in Nude Mice Bearing the KRAS_G12D Mutant AsPC-1 Human Pancreatic Tumor Xenografts

To investigate the antitumor efficacy of Compound (1) against KRAS G12D mutated AsPC-1 human pancreatic tumors, 40 and 80 mg/kg/day of Compound (1), 1 mg/kg/day of trametinib, and the combination of 80 mg/kg/day Compound (1) and 1 mg/kg/day trametinib have been administered to male BALB/c nude mice subcutaneously implanted with AsPC-1 tumor xenograft for 3 weeks. All treatments were administered every day by oral gavage. The tumor volumes and body weights of animals were recorded.

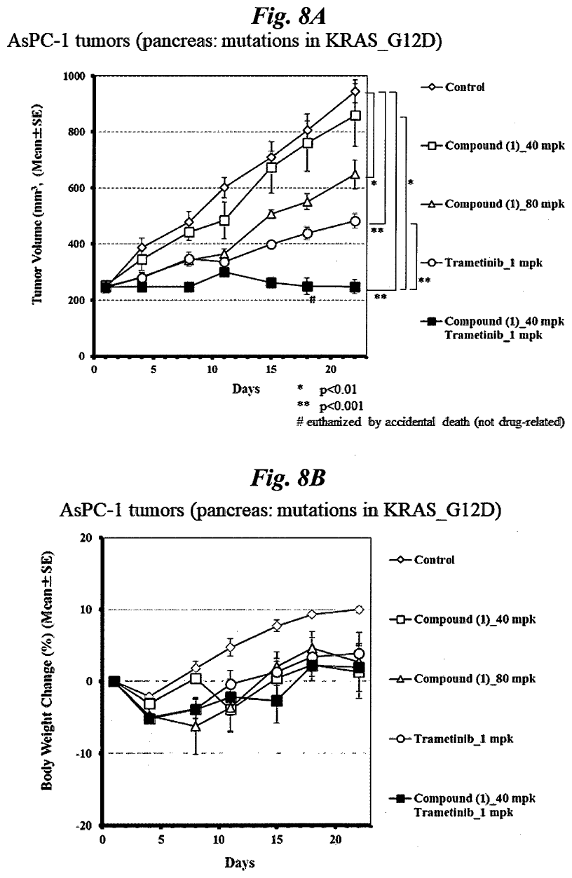

The results are summarized in FIGS. 8A-8B and Table 6 (evaluated at Day 22).

TABLE 6
Antitumor effect of Compound (1) and Trametinib in mice with AsPC-1 xenograft tumors

| | | | | | | | | p-value | | | |
|---|---|---|---|---|---|---|---|---|---|---|---|
| | Dose | | | Number | Number | Tumor volume (mm³) | | | vs | | T/C |
| | (mg/kg/ | Dosing | | of | of | Day 1 | Day 22 | vs | Compound | vs | ratio |
| Group | day) | schedule | Route | animals | deaths | Mean ± SE | Mean ± SE | control | (1)_40 | Trametinib | (%) |
| Control | — | — | — | 5 | 0 | 243.0 ± 15.2 | 943.9 ± 40.6 | — | — | — | 100 |
| Compound (1) | 40 | qd, Day 1-21 | po | 5 | 0 | 252.8 ± 16.6 | 860.1 ± 112.1 | n.s. | — | — | 91 |
| Compound (1) | 80 | qd, Day 1-21 | po | 5 | 0 | 243.6 ± 17.9 | 648.0 ± 51.7 | p < 0.01 | — | — | 69 |
| Trametinib | 1 | qd, Day 1-21 | po | 5 | 0 | 248.3 ± 16.0 | 482.5 ± 25.6 | p < 0.001 | — | — | 51 |
| Compound | 40 + 1 | qd, Day 1-21 + | po + po | 4* | 0 | 246.5 ± 11.0 | 248.1 ± 24.5 | p < 0.001 | p < 0.01 | p < 0.01 | 26 |
| (1) + | | qd, Day 1-21 | | | | | | | | | |
| Trametinib | | | | | | | | | | | |

Abbreviations: qd, quaque die (once daily);
po, per os (orally);
SE, standard error; T/C ratio,
treatment/control ratio of the mean tumor volume;
NA, not applicable
Note:
Tumor volume (mm3) = (major axis [mm]) × (minor axis [mm]) × (minor axis [mm])/2
T/C ratio (%) = (mean tumor volume of each treated group on Day 22)/(mean tumor volume of the control group on Day 22) × 100
Compound (1) and Trametinib afatinib were administered orally once daily for 21 days.
Statistical analysis was performed with Dunnett's test to compare the Day 22 tumor volume of each treated group with that of the control group.
*Animal No. 3 Accidental Death

Trametinib showed significant inhibition of tumor growth (T/C: 51%, p<0.001), whereas Compound (1) showed a limited antitumor effect in both 40 and 80 mg/kg/day groups (T/C: 91%, not significant, T/C: 69%, p<0.01, respectively). Notably, a significant combination effect (T/C: 26%, p<0.001 versus trametinib alone, p<0.01 versus 40 mg/kg/day of Compound (1) group) was observed. Regarding Compound (1) administered groups, there were no significant changes in weight reduction or tolerability, although a non-drug related death was observed on Day 18 due to administration error.

### Example 11: Evaluation of Growth Inhibitory Activity of Compound (1), Trametinib, and AMG510 Against KRAS Mutant Cell Lines Used in Animal Experiments

The IC50 values for each compound in cell lines used in a series of animal experiments evaluating the antitumor effects of Compound (1) on KRAS mutant tumor xenografts were determined by a 3-day growth inhibition assay (Table 7).

TABLE 7

| | Growth inhibition (IC₅₀: nmol/L) | | | |
|---|---|---|---|---|
| | SW1573 | LU65 | LS180 | AsPC-1 |
| | (NSCLC) | (NSCLC) | (Colon) | (Pancreas) |
| Compound (1) | 139 | 92.9 | 316 | 184 |
| Trametinib | >10,000 | 0.739 | 9 | 0.693 |
| AMG510 | >10,000 | 7.17 | >10,000 | n.t. |

Genetic alteration

| | | | | |
|---|---|---|---|---|
| KRAS | p.G12C | p.G12C | p.G12D | p.G12D |
| PIK3CA | p.K111E | WT | p.H1047R | WT |
| PTEN | WT | WT | p.I67K | WT |

Compound (1) showed a relatively stable growth inhibitory effect on KRAS mutant cancer cell lines, but the growth inhibitory effects of trametinib and AMG510 were significantly attenuated in cells with mutations in PIK3CA and PTEN genes (SW1573 and LS180). These findings are consistent with the relative response to Compound (1) and AMG510 and trametinib observed in efficacy studies.

Obviously, numerous modifications and variations of the present invention are possible in light of the above teachings. It is therefore to be understood that within the scope of the appended claims, the invention may be practiced otherwise than as specifically described herein.

1-27. (canceled)

28. A method of treating a subject with a cancer having an aberration in PTEN, the method comprising:
administering to the subject an effective amount of 4-(4-(3-((2-(tert-butylamino)ethyl)amino)-6-(5-(trifluoromethyl)-1,3,4-oxadiazol-2-yl)pyridin-2-yl)piperidin-1-yl)-5,5-dimethyl-5H-pyrolo[2,3-d]pyrimidin-6(7H)-one (TAS0612) or a pharmaceutically acceptable salt thereof.

29. The method of claim 28, wherein the cancer is at least one selected from the group consisting of breast cancer, thyroid cancer, renal cell carcinoma, endometrial cancer, colorectal cancer, melanoma, glioblastoma, prostate cancer, ovarian cancer, and lung cancer.

30. The method of claim 28, wherein the cancer is endometrial cancer.

31. The method of claim 28, wherein the subject is determined to have the aberration in PTEN prior to administering TAS0612 or a pharmaceutically acceptable salt thereof.

32. The method of claim 28, wherein the cancer has a co-occurring aberration in PTEN and at least one selected from the group consisting of KRAS, BRAF, PIK3CA, AKT1, EGFR, HER2, TP53, NF1, and BRCA.

33. The method of claim 28, wherein the cancer has a co-occurring aberration in PTEN and PIK3CA.

34. The method of claim 28, wherein the aberration in PTEN is PTEN loss of gene or variants which lead to loss of function.

35. The method of claim 28, wherein TAS0612 or a pharmaceutically acceptable salt thereof is administered orally to the subject.

36. The method of claim 28, wherein TAS0612 or a pharmaceutically acceptable salt thereof is administered to the subject once per day (QD).

37. The method of claim 28, wherein from about 10 to about 960 mg of TAS0612 or a pharmaceutically acceptable salt thereof is administered to the subject per day.

38. The method of claim 28, wherein TAS0612 or a pharmaceutically acceptable salt thereof is administered daily to the subject for at least 28 days.

39-49. (canceled)
